SEQUENCE LISTING (6 sequences)

SEQ ID NO: 1 - RNA, 25 nt, Artificial Sequence
  alpha globin mRNA synthetic 25-mer
ccaguccgucugagaaggaaccacc

SEQ ID NO: 2 - DNA, 20 bp, Artificial Sequence
  antisense
acgttgaggggcatcgtcgc

SEQ ID NO: 3 - RNA, 25 nt, Artificial Sequence
  antisense
ggugguuccuucucagacggacugg

SEQ ID NO: 4 - RNA, 22 nt, Artificial Sequence
  antisense
ugagagcugaaagcagguccau

SEQ ID NO: 5 - RNA, 22 nt, Artificial Sequence
  antisense
uaccuggacgaaagucgagagu

SEQ ID NO: 6 - DNA, 22 bp, rat
actctcgactttcgtccaggta

(19) United States
(12) Patent Application Publication
(10) Pub. No.: US 2003/0171328 A1
(43) Pub. Date: Sep. 11, 2003

(54) Non-invasive method for detecting target-RNA

(75) Inventor: Patrick L. Iversen, Corvallis, OR (US)

Correspondence Address: PERKINS COIE LLP, P.O. BOX 2168, MENLO PARK, CA, 94026, US

(73) Assignee: AVI BioPharma, Inc.

(21) Appl. No.: 10/421,173

(22) Filed: Apr. 22, 2003

Related U.S. Application Data
(62) Division of application No. 09/736,920, filed on Dec. 13, 2000, now abandoned.
Application No. 09/736,920 is a continuation-in-part of application No. 09/493,494, filed on Jan. 28, 2000, now Pat. No. 6,365,351.
(60) Provisional application No. 60/117,846, filed on Jan. 29, 1999.

(51) Int. Cl. C12Q 1/68; G01N 33/53; A61K 48/00

(52) U.S. Cl. 514/44; 435/6; 435/7.1

(57) ABSTRACT

A method of detecting in a subject, the occurrence of a base-specific intracellular binding event involving a single-stranded target RNA, is disclosed. The method includes administering to the subject an oligomeric antisense compound having (i) from 8 to 40 bases, including a targeting base sequence that is complementary to a portion of the target RNA, (ii) a Tm, with respect to binding to a complementary RNA sequence, of greater than about 50° C., and (iii) an ability to be actively taken up by mammalian cells, and (iv) conferring resistance of complementary RNA hybridized with the agent to RnaseH. Where the compound is administered in uncomplexed form, it preferably has a substantially backbone. At a selected time after said administering the agent, a sample of a body fluid is obtained from the subject, and the presence in the sample of a nuclease-resistant heteroduplex composed of the antisense oligomer and the complementary portion of the target RNA is detected. The method is useful, for example, for detecting levels of gene expression, biochemical or physiological states that are characterized by expression of certain genes, genetic mutations, and the presence and identity of infective viral or bacterial agents. Also disclosed are arrays, kits and antibodies employed in carrying out the method.

This application is a continuation-in-part of U.S. patent application Ser. No. 09/493,494, filed Jan. 28, 2000, which claims priority to U.S. Provisional Application Serial No. 60/117,846, filed Jan. 29, 1999, now abandoned, both of which are incorporated by reference herein.

### FIELD OF THE INVENTION

The present invention relates to a non-invasive method for detecting the presence of RNA target sequences in vivo, and to arrays, kits and antibodies useful in practicing the method.

### REFERENCES

Bloomers, M., Nuc Acids Res, 22(20):4187 (1994)

Brown, S. et al., Science 265:777 (1994).

Cross, V., et al., Biochem, 36:4096 (1997)

Cruse, W. et al., J. Mol Biol, 192:891 (1986).

Dagle, et al., Nuc Acids Res, 28(10): 2153 (2000).

Ding, D. et al., Nuc Acids Res, 24(2):354 (1996).

Gait, M. J.; Jones, A. S. and Walker, R. T. (1974) J. Chem. Soc. Perkin I, 1684-1686.

Heinemann, U., et al., Nuc Acids Res, 19:427 (1991)

Huie, E. M.; Kirshenbaum, M. R.; and Trainos, G. L. (1992) J. Org. Chem. 57:4569.

Jones, A. S.; MacCross, M. and Walker, R. T. (1973) Biochem. Biophys Acta 365:365-377.

Kang, H.; Chou, P. J.; Johnson, W. C.; Weller, D.; Huang, S. B;

Summerton, J. E. (1992), Biopolymers 32:1351-63.

Lacroix, L., et al., Biochem Biophys Res Commun, 270:363 (2000).

Matteucci, M. (1990) Tetrahedron Left. 31:2385-2388

McElroy, E. B.; Bandarn, R.; Huand, J.; and Widlanski, T. S. (1994) Bioorg. Med. Chem. Lett. 4:1071.

Mertes, M. P. and Coates, E. A. (1969) J. Med. Chem. 12:154-157.

Miller, P.S., Ts'O, P.O.P.; Hogrefe, R. I.; Reynolds, M. A.; and Arnold, L. J. (1993) In: Antisense Research Applications. Crooke, S. T. and Lebleu, B. (eds): Boca Raton: CRC Press, pp. 189.

Mohan, V. et al., Tetrahedron, 51:8655 (1995).

Neilsen, P. E.; Engholm, M.; Berg, R. H.; and Buchardt, O. (1991) Science 254:1497-1500

Olgive, K. K. and Cormier, J. F. (1986) Tetrahedron Lett 26:41594162.

Piotto, M. et al., Tetrahedron, 47:2449 (1991)

Ray, A., et al., FASEB 14:1041 (2000)

Roughton; A. L., Portman, S.; Benner, S. A.; and Egli, M. (1995) J. Am. Chem. Soc. 117:7249.

Schroeder, H. W., “Part IV, Principles of Human Genetics”, Chapter 23 in Cecil Textbook of Medicine (ed. J. Claude Benett and F. Blum) 20th ed.

W. B. Saunders Co., Philadelphia, 1996, pp. 134-165

Stein, C. et al., Pharmacol & Therapeutics, 85:231 (2000).

Stirchak, E., et al., Nuc Acids Res, 17(15):6129 (1989).

Vasseur, J. J.; Debart, F.; Sanghvi, Y. S.; and Cook, P. D. (1992) J. Am. Chem. Soc. 114:4006.

### BACKGROUND OF THE INVENTION

Diagnosis and monitoring of various disease conditions is accomplished by analyzing peptides, proteins, antibodies and/or nucleic acids associated with the condition.

In recent years, analysis of gene and other genomic sequences has become an important tool for identifying genetic diseases or predisposition to such diseases, and for monitoring levels of gene expression that are characteristic of particular pathologies or cells types, or in response to drugs aimed at modulating functional gene expression. Currently, genetic analyses of this type are carried out ex vivo, typically by obtaining a tissue of blood sample from an individual, and analyzing genomic DNA, cDNA or mRNA for the presence of absence of certain sequence mutations or for elevated or depressed levels of gene expression, or for viral- or bacterial-specific sequences.

Diagnostic devices, e.g., gene chips, for detecting mutations or changes in level of expression are now available, with new capabilities under development. Similar methods may be employed to monitor the effect of therapeutic compounds on gene expression in individuals. That is, following compound administration, a tissue biopsy or blood sample may be obtained from the treated patient to determining the effect of the compound on expression of one or more targeted genes.

Although analysis of mutations and levels of gene expression by these in vitro methods has the capability of yielding important information about the gene makeup and the drug response of an individual, the methods are often impractical, expensive and/or unable to provide the desired information. For example, it is generally not practical to biopsy an individual's tissue to monitor gene expression in that tissue, both because of the difficulty and risk to patient of obtaining a tissue sample, and because of the expense of working up a tissue sample for analysis.

It would therefore be highly desirable to be able to detect gene mutations and monitor levels of gene expression, or gene expression in response to therapeutic agents by methods that do not require obtaining tissue or cellular samples from an individual, or isolating and measuring nucleic acids samples obtained from such cells or tissue.

One of the therapeutic approaches for modulating mRNA levels in cells that has been proposed is antisense therapy. Typically, the approach employs a nucleic acid or nucleic acid analog capable of binding by Watson-Crick base pairing to a known-sequence region of the target mRNA, e.g., a region spanning the mRNAs start codon or a splice junction site. If the antisense compound is able to find and enter target-tissue cells, and inactivate mRNA processing or translation, it should be effective in reducing functional expression products of the mRNA, and thus produce a desired therapeutic effect.

It would be further desirable, in antisense therapy, to confirm that the antisense compound administered is being taken up by cells and binds to (and therefore presumably inactivates) target mRNA molecules.

Another diagnostic application of gene-sequence analysis is in identifying viral or bacterial agents in an infected subject. The analysis may even extend to identifying the presence of levels of expression of antibiotic-resistant genes, for purposes of deciding on the most effective course of treatment. Such gene-sequence analysis, however, typically requires either laborious culture and/or PCR techniques. It would be desirable, therefore, to provide a method of analyzing viral or bacterial (or fungal) infective agents by a simple, relatively fast assay method.

### SUMMARY OF THE INVENTION

The invention includes, in one aspect, a method of detecting in a subject, the occurrence of a base-specific intracellular binding event involving a single-stranded target RNA. In practicing the method, there is administered to the subject, an oligomeric antisense compound having (i) from 8 to 40 bases, including a targeting base sequence that is complementary to a portion of the target RNA, (ii) a Tm, with respect to binding to a complementary RNA sequence, of greater than about 50° C., and (iii) an ability to be actively taken up by mammalian cells, and (iv) conferring resistance of complementary mRNA hybridized with the agent to RNases, such as RnaseH, capable of cutting RNA in double-stranded form. Preferably, the agent has a substantially uncharged backbone, or is complexed with a compound, e.g., polycation, that renders the complex suitable for active uptake, e.g., by endocytosis, into cells. At a selected time after the compound is administered, a sample of body fluid from the patient is obtained, and the sample is analyzed to detect the presence of a nuclease-resistant heteroduplex composed of the antisense oligomer and the complementary portion of the RNA transcript.

In various preferred embodiments, the antisense compound is a morpholino antisense compound having uncharged, phosphorous-containing intersubunit linkages, as exemplified by compounds (A)-(D) shown in FIG. 5.

The detecting step may include capturing the heteroduplex on a solid support, by contacting the duplex with a support-bound capture agent capable of binding heteroduplex but not the free antisense agent, and detecting heteroduplex so captured. Preferred capture agents are: (a) an antibody capable of binding in a sequence-independent manner to the heteroduplex, (b) an antibody capable of binding in a sequence-dependent manner to a heteroduplex in a sequence-dependent manner, (c) an antibody capable of binding to an antigen attached to the antisense compound, (d) a non-antibody antiligand molecule capable to binding to a ligand moiety attached to the antisense compound, and (e) a base-specific duplex-binding oligomer.

In one general embodiment, the presence of heteroduplex on the solid support is detected by placing the support and bound heteroduplex in contact with a labeled (detectable) heteroduplex-binding agent, such as a labeled antibody. In another general embodiment, the support-bound heteroduplex is eluted from the support and detected in a released form, e.g., by electrophoresis or mass spectroscopy.

For use in detecting changes in expression of a target gene in response to a therapeutic agent in the subject, the target RNA is mRNA produced by expression of the target gene, the steps of the invention are performed at a selected times before and administration of the therapeutic agent, and the levels of heteroduplex before and after such administration are compared.

For use in detecting the presence or levels of an mRNA which is diagnostic of a given biochemical or pathological condition or a predisposition to such condition, such as (i) pregnancy, (ii) heart disease, (iii) alcoholism, and (iv) cancer, the target RNA is an mRNA encoding a protein diagnostic of the selected condition.

For use in detecting the presence of a mutated gene which is diagnostic of a given genetic disease, the target RNA is an mRNA transcribed by the gene and encodes a mutated protein characteristic of a genetic disease. The antisense compound may be designed to form a stable heteroduplex above 50° C. only with the mutated form of the mRNA, and the detecting step may optionally include heating heteroduplex in the sample above a selected temperature, e.g., 50° C., to denature heteroduplexes having one or more internal-base mismatches.

For use in detecting the presence of an infective viral or bacterial agent in the subject, the target RNA is a single-stranded RNA or DNA having a virus-specific or bacteria-specific sequence, respectively.

In one embodiment, the antisense agent is administered by applying the agent to a region of the subject's skin, the body sample is obtained by applying an adhesive tape to the skin region, and the presence of heteroduplex in the sample is detected by assaying the tape for the presence of bound heteroduplex.

In another aspect, the invention includes a method of detecting in a subject, the occurrence of base-specific intracellular binding events involving a plurality of target RNAs. The method differs from the above-described method in that a plurality of different-sequence oligomeric antisense compounds are administered to the patient, and the detecting steps is applied to the plural heteroduplex species that may form.

Where the heteroduplex species are detected on a solid support, the support may have an array an array of regions, where each region contains a sequence-specific support-bound capture agent capable of specifically binding to a heteroduplex species of a selected sequence. The capture agent in the array may be, for example, (a) an antibody capable of binding in a sequence-dependent manner to a heteroduplex, (b) an antibody capable of binding to a sequence-specific antigen attached to the antisense compound, or (c) a sequence-specific duplex-binding oligomer.

Where the heteroduplex are detected in solute or suspension form, the heteroduplex species are preferably first isolated by binding to a solid support, and after release from the support are detected by methods capable of distinguishing different-sequence species, e.g., by electrophoresis or mass spectroscopy, where the different-sequence heteroduplexes have different molecular weights and/or charges.

For use in detecting one of a plurality of different known-mutation gene sequences associated with one or more known disease states, the target RNAs are mRNA's transcribed by the gene sequences and encode mutated proteins associated with selected genetic diseases.

For use in detecting the presence of one or more of a plurality of different viruses or bacteria, the steps in the method may be carried out successively using first and second sets of antisense agents effective to bind to viral or bacterial sequences representing relatively broad and relatively narrow classes of viruses or bacteria, respectively. The second set of antisense agents is selected on the basis of the heteroduplex(es) formed and detected using the first set of agents.

In another embodiment, which uses a skin assay system in accordance with the invention, the antisense agents are administered by applying the subject's skin, an adhesive pad containing a lower adhesive layer adapted to be attached adhesively to the subject's skin, and defining an array of holes adapted to expose an array of skin regions, and a removable antisense delivery layer containing an array of different-sequence antisense agents at positions corresponding to the lower-layer holes. The detecting step involves removing the delivery layer and replacing it with an adhesive sample-collection layer, to collect sample on the adhesive layer at array regions corresponding to the holes. The array of samples is then assayed for the presence of heteroduplex at each array region.

Also forming a part of the invention is a diagnostic array device for detecting in a subject, the occurrence of base-specific intracellular binding events involving a plurality of target RNAs. The array device includes a substrate divided into a plurality of regions. Carried on each array region is a sequence-specific binding agent capable of binding to a specific-sequence RNA/antisense heteroduplex of the type described above. Also disclosed is a kit containing the array device and a detection reagent capable of binding to such heteroduplex species bound to one or more regions of the array.

In still another aspect, the invention includes a monoclonal antibody having specific binding affinity for an oligomer:RNA heteroduplex of the type described above. The binding affinity of the antibody for the heteroduplex may be substantially independent of heteroduplex sequence, or may be require a specific sequence in a region of the heteroduplex.

These and other objects and features of the invention will become more fully apparent when the following detailed description is read in conjunction with the accompanying drawings.

### BRIEF DESCRIPTION OF THE DRAWINGS

FIG. 1 shows interactions of an antisense molecule in forming a heteroduplex that is excreted from a body, consistent with the in vivo data obtained in accordance with the method of the invention;

FIG. 2 is a plot of the disappearance of a P450 antisense phosphorodithioate morpholino oligomer (PMO) and appearance of PMO:mRNA heteroduplex in the plasma of rats administered the over time (minutes), where the open boxes correspond to PMO and the closed circles correspond to PMO″RNA duplex;

FIG. 3 shows a variety of antisense molecules with uncharged backbones that are candidate molecules for use in the invention;

FIG. 4 shows one class of preferred antisense subunits having various linking groups suitable for forming antisense compounds suitable for use in the invention;

FIGS. 5A-D show the repeating subunit segment of exemplary morpholino oligonucleotides, designated A through D/E, constructed using subunits A-E, respectively, of FIG. 4;

FIGS. 6A-6E illustrate various types of solid-support/heteroduplex interactions that be employed in detecting heteroduplex species in accordance with the invention;

FIGS. 7A-7C illustrate various types of detection reagents used to in detecting a heteroduplex on a solid support in accordance with one embodiment of the invention;

FIGS. 8A-8D illustrate steps in detecting a heteroduplex in a purified solution form, in accordance with another embodiment of the invention, where purified or partially purified heteroduplexes are assayed by mass spectroscopy (8C) or gel electrophoresis (8D);

FIG. 9 shows a portion of an array device formed in accordance with an aspect of the invention;

FIG. 10 illustrates a hypothetical test result to determine the presence of each of a plurality of mRNAs species in a subject; and

FIG. 11 is a plan view of a transdermal array applicator employed in a transdermal embodiment of the invention;

FIG. 12 is an enlarged sectional device of the applicator FIG. 11, taken along view line 12-12;

FIG. 13 is a sectional view of an array collector employed in the transdermal embodiment of the invention; and

FIG. 14 is a plan view of a multi-well detection device used in the transdermal embodiment.

### DETAILED DESCRIPTION OF THE INVENTION

I. Definitions

The terms below, as used herein, have the following meanings, unless indicated otherwise:

As used herein, the term “oligonucleotide” is used interchangeably with the term “antisense oligonucleotide”, “antisense agents”, “antisense compound”, and “antisense oligomer” and to refer to an nucleotide-analog oligomer having a sequence of nucleotide bases and a subunit-to-subunit backbone linkages that allows the antisense oligomer to hybridize to a target sequence in an RNA by Watson-Crick base pairing, to form an oligomer:RNA heteroduplex within the target sequence. The oligomer may have exact sequence complementarity to the target sequence or near complementarity. These antisense oligomers may block or inhibit translation of the mRNA containing the target sequence, or block mRNA processing, e.g., slice-junction processing, or inhibit gene transcription, where the oligonucleotide is a double-stranded binding agent. The terms “compound”, “agent”, “oligomer” and “oligonucleotide” may be used interchangeably with respect to the antisense oligonucleotides of the invention.

As used herein, the term “antisense oligomer composition” refers to a composition comprising one or more antisense oligomers for use in the RNA detection methods of the present invention. In some cases, such an “antisense oligomer composition” contains a plurality of antisense oligomers.

As used herein, a “morpholino oligomer” refers to an antisense oligomer having a backbone which supports bases capable of hydrogen bonding to typical polynucleotides, where the polymer backbone moiety is a morpholino group rather than a pentose sugar.

As used herein, the term “PMO” refers to a phosphordiamidate morpholino oligomer, as further described below, wherein the oligomer is a polynucleotide of about 840 bases in length, preferably 12-25 bases in length. This preferred aspect of the invention is illustrated in FIG. 5B, which shows two such subunits joined by a phosphorodiamidate linkage.

As used herein, a “nuclease-resistant” oligomeric molecule (oligomer) is one whose backbone is not susceptible to nuclease cleavage of a phosphodiester bond. Exemplary nuclease resistant antisense oligomers are oligonucleotide analogs such as methyl-phosphonate, morpholino, and peptide nucleic acid (PNA) oligonucleotides, all of which have uncharged backbones.

As used herein, an oligonucleotide or antisense oligomer “specifically hybridizes” to a target polynucleotide if the oligomer hybridizes to the target under physiological conditions, with a Tm substantially greater than 37° C., preferably at least 50° C., and typically 60° C.-80° C. or higher. Such hybridization preferably corresponds to stringent hybridization conditions, selected to be about 10° C., and preferably about 5° C. lower than the thermal melting point (Tₘ) for the specific sequence at a defined ionic strength and pH. At a given ionic strength and pH, the Tₘ is the temperature at which 50% of a target sequence hybridizes to a complementary polynucleotide.

Polynucleotides are described as “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree that one polynucleotide is complementary with another) is quantifiable in terms of the proportion of bases in opposing strands that are expected to form hydrogen bonds with each other, according to generally accepted base-pairing rules.

As used herein, a first sequence is an “antisense sequence” with respect to a second sequence if a polynucleotide whose sequence is the first sequence specifically binds to a polynucleotide whose sequence is the second sequence.

As used herein, a “base-specific intracellular binding event involving a target RNA” refers to the specific binding of an antisense oligomer with a complementary target RNA sequence inside a cell.

As used herein, “nuclease-resistant heteroduplex” refers to a heteroduplex formed by the binding of an antisense oligomer to its complementary target, in which both the antisense and the complementary region of the RNA are resistant to in vivo degradation by intracellular and extracellular nucleases.

As used herein, the term “target”, relative to an mRNA or other RNA species, e.g., viral genomic RNA, refers to an mRNA or other RNA which is expressed or present in single-stranded in one or more types of mammalian cells. Preferentially expressed means the target mRNA is derived from a gene expressed in to a greater extent in one cell type than another.

As used herein, “effective amount” relative to an antisense oligomer refers to the amount of antisense oligomer administered to a mammalian subject, either as a single dose or as part of a series of doses, that is effective to specifically hybridize to all or part of a selected target sequence forming a heteroduplex between the target RNA and the antisense oligomer which may subsequently be detected in a body fluid of the subject.

As used herein, the term “body fluid” encompasses a variety of sample types obtained from a subject including, urine, saliva, plasma, blood, spinal fluid, or and other sample of biological origin, such as skin cells or dermal debris, and may refer include cells or cell fragments suspended therein, or the liquid medium and its solutes.

The term “relative amount” is used where a comparison is made between a test measurement and a control measurement. The relative amount of a reagent forming a complex in a reaction is the amount reacting with a test specimen, compared with the amount reacting with a control specimen. The control specimen may be run separately in the same assay, or it may be part of the same sample (for example, normal tissue surrounding a malignant area in a tissue section).

An antisense agent has “an ability to be actively taken up by mammalian cells” is the agent can enter the cell by a mechanism other than passive diffusion across the cell membrane. The agent may be transported, for example, by “active transport”, referring to transport agents across a mammalian cell membrane by an ATP-dependent transport mechanism or by “facilitated transport”, referring to transport of antisense agents across the cell membrane by a transport mechanism that requires binding of the agent to a transport protein, which then facilitates passage of the bound agent across the membrane. For both active and facilitated transport, the antisense agent has a substantially uncharged backbone, as defined below. Alternatively, the antisense compound may be formulated in a complexed form, such as an agent having an anionic backbone complexed with cationic lipids or liposomes, which can be taken into cells by an endocytotic mechanism.

II. Method of the Invention

The present invention is based on the discovery that certain antisense compounds, when administered to a mammalian subject, subsequently appear in the urine (or other body fluid) in the form of a duplex of the antisense and the complementary portion of target RNA. The observations underlying the discovery are illustrated in Examples 1 and 2.

The study in Example 1 shows that an antisense agent (in this case, a PMO) hybridizes with a complementary RNA target to form a nuclease resistant duplex that migrates more slowly than the associated single-strands RNA (ssRNA) on gel electrophoresis, presumably due to the greater mass/charge ratio of the duplex.

Also in Example 1, an antisense oligomer agent (PMO) was injected IP in mammals, and 24 hours later, a urine sample was taken. After treatment of the sample with RNases (e.g., 3′- and 5-exonucleases), the nucleic acids in the sample were analyzed by gel electrophoresis. The results show the presence of a band that migrates with the migration rate of an oligomer:RNA heteroduplex (as studied in Example 1). Appearance of the duplex band is dose dependent.

Example 2 examines the time course of appearance of the antisense/RNA duplex following antisense administration to a mammalian subject (in this case, a rat). Blood samples were taken at times 0, 1, 2, 4, 8, 12, and 24 hours following injection of an antisense against rat P450. Electrophoretic migration times and mass spectral analysis of fluorescence-labeled species in the blood sample are both consistent with an oligomer:RNA duplex.

The measured levels of the labeled antisense (open squares) and oligomer:RNA heteroduplex (closed circles) in the blood samples is shown in FIG. 2. Levels of labeled antisense quickly decline in the bloodstream within two hours after injection. The duplex appears in the blood between 4-8 hours post injection, and peaks sometime between 8 and 24 hours.

IIA. Model of Duplex Formation

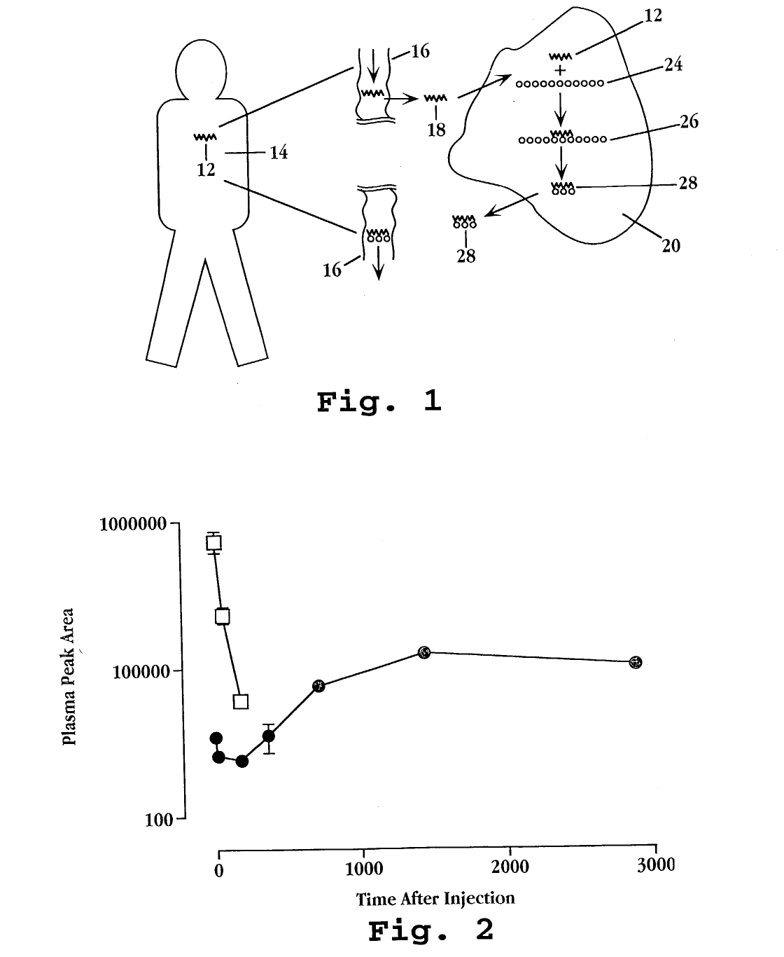

Taken together the data point to, and are consistent with a model of antisense uptake and processing illustrated in FIG. 1. Initially, an antisense agent 12 is administered to a subject 14, e.g., by oral, IV, IM, subQ, or transdermal administration. The compound makes its way to the bloodstream, shown at 16, and from there, is distributed to an extracellular space 18 bathing cells, such as cell 20. The compound is taken up, preferably by active or facilitated transport, into the cell, where it hybridizes with the complementary region of a target RNA 24, forming an oligomer:RNA heteroduplex 26. The single-strand (non-hybridized) RNA regions of the duplex are susceptible to RNase degradation, and may be enzymatically cleaved, partially or completely, within the cell or after expulsion from the cell, to form an oligomer:RNA heteroduplex 28 with little or no single-strand overhang. The duplex, being recognized as a “foreign” species is then expelled from the cells into the surrounding extracellular space, and from there, back into the bloodstream, where the duplex may be cleared, for example, into the urine.

The data above indicate that the period required for uptake and processing of the duplex into its duplex form occurs in the period 8-24 hours post injection.

IIB. Selection of Antisense Agents

The model above imposes four basic requirements on the antisense compound employed in the invention, considered in the five subsections below.

B1. Selected target sequence. The antisense compound must be targeted, in base sequence, against a selected RNA target sequence. Antisense compounds whose region of complementarity with the target RNA sequence may be as short as 10-12 bases, but are preferably 13-20 bases. Antisense oligonucleotides of 15-20 bases are usually long enough to have one complementary sequence in the mammalian genome. In addition, a minimum length of complementary bases may be required to achieve the requisite binding Tm, as discussed below. Oligomers as long as 40 bases may be suitable, where at least the minimum number of bases, e.g., 10-15 bases, are complementary to the target RNA sequence, but in general, facilitated or active uptake in cells is optimized at oligomer lengths less than about 20 bases.

The target RNA sequence generally will fall into one of five different classes of RNA of interest: (i) genes whose expression is to be inhibited by a therapeutic antisense, e.g., c-myc or p53 antisense; (ii) genes whose expression indicates at given biochemical state, e.g., pregnancy, liver ALT, or markers for heart-associated pathologies; (iii) genetic mutations, diagnostic of genetic diseases or a predisposition to same; (iv) viral genomic sequences corresponding to viruses capable of infecting humans and other mammals, e.g., veterinary animals, and (v) bacterial (or fungal) genomic sequences corresponding to bacteria (or fungi) capable of infecting humans of other mammals. Target RNA sequences for each of these five classes are considered in detail in Section D below.

B2. High Tm. The oligomer compound must form a stable hybrid duplex with the target sequence. The antisense compound will have a binding Tm, with respect to a complementary-sequence RNA of greater than body temperature and preferably greater than 50° C. Tm's in the range 60-80° C. or greater are preferred. The Tm of an antisense compound with respect to complementary-sequence RNA may be measured by conventional methods, such as those described by Hames et al., Nucleic Acid Hybridization, IRL Press 1985, p. 107-108. According to well known principles, the Tm of an oligomer compound, with respect to a complementary-base RNA hybrid, can be increased by increasing the ratio of C:G paired bases in the duplex, and/or by increasing the length (in basepairs) of the heteroduplex. At the same time, for purposes of optimizing cell transport, it may be advantageous to limit the size of the oligomer. For this reason, compounds that show how Tm (50° C. or greater) between 15-20 bases or less will be preferred over those requiring 20+ bases for high Tm values.

B3. Active uptake by cells. In order to achieve adequate intracellular levels, the antisense oligomer must be taken be actively taken up by cells, meaning that the compound is taken up by facilitated or active transport, if administered in free (non-complexed) form, or is taken by an endocytotic mechanism if administered in complexed form.

In the case where the agent is administered in free form, the agent should be substantially uncharged, meaning that a majority of its intersubunit linkages are uncharged at physiological pH. Alternatively, the oligomer may contain both negatively and positively charged backbone linkages, as long as two opposite charges are substantially offsetting, and preferably do not include runs of more than 3-5 subunits or either charge. For example, the oligomer may have a given number of anionic linkages, e.g., N3-P5 phosphoramidate linkages, and a comparable number of cationic linkages, such as N,N, diethylelene-diamine phosphoramidates (Dagle).

Preferably the number of charges (or the net charge) is no more than 1 charge group per five subunits. Experiments carried out in support of the invention indicate that a small number of changes, e.g., 1-2, may actually enhance cell uptake of certain oligomers with uncharged backbones. The charges may be carried on the oligomer itself, e.g., in the backbone, or may be terminal charged-group appendages.

In addition to being uncharged, the antisense agent should be a substrate for a membrane transporter system (membrane protein or proteins) capable of facilitating transport or actively transporting the oligomer across the cell membrane. This letter feature may be determined by one of a number of tests for oligomer interaction or cell uptake.

A first test examines the ability of an oligomer compound to displace or be displaced by a selected oligomer, e.g., phosphorothioate oligomer on a cell surface. For purposes of the test, either a mammalian cell in culture or a bacterial cell may be employed as the cell substrate. The cells are initially incubated with a given quantity of test agent, e.g., fluorescence-labeled test agent, at a final oligomer concentration of between about 10-300 nM. Shortly thereafter, e.g., 10-30 minutes (before significant internalization of the test compound can occur), a second oligomer compound, e.g., a phosphorothioate oligomer of the same sequence, known to bind specifically to cell receptor (displacing compound) is added, at each of a number of increasing concentrations. If the test compound binds specifically to the cell receptor, it will be displaced by the displacing compound, in a concentration-dependent manner. If the displacing compound is able to produce 50% displacement at a concentration of 10× the test compound concentration or less (typically 0.5 to 2×) are considered to have adequate binding at the same recognition site for the cell transport system.

A second test for cell transport directly examines the ability of the test compound to transport a labeled reporter, e.g., a fluorescence reporter, into cells. Again the cell substrate may be a bacterial or cultured mammalian. The cells are incubated in the presence of labeled test compound, added at a final concentration preferably between about 10 to 300 nM. After incubation for 30-120 minutes, the cells are examined, e.g., by microscopy, for intracellular label. The presence of significant intracellular label is evidence that the test compound is transported by facilitated or active transport.

A third test relies on the ability of certain antisense compounds to effectively inhibit bacterial growth, when target against bacterial 16S rRNA observed. Studies carried out in support of the present invention show that the inhibition requires active or facilitated transport across cell (in this case, bacterial cell) membranes. The test compound is prepared with a target 16S sequence, such as SEQ ID. NOS: 1-3, which are representative sequences against E. coli 16S rRNA that have been demonstrated to be effective in inhibiting bacterial growth. The compound is added to a growing bacterial culture, e.g., E. coli culture, at increasing concentrations, typically between 10 nM and 1 mM. The ability to inhibit bacterial growth is measured from number of cell colonies cell counts at 24-72 hours after addition of the test compound. Compounds which can produce a 50% inhibition at a concentration of between about 100-500 nM or lower are considered to be good candidates for active transport in mammalian cells.

In the second case, where the antisense compound is administered in a complexed form, the agent may have a charged, e.g., anionic backbone, where the complexing agent typically is a polymer, e.g., cationic lipid, polypeptide, or non-biological cationic polymer, having an opposite charge. Methods of forming complexes, including bilayer complexes, between anionic oligonucleotides and cationic lipid or other polymer components are well known, and applicable to the present invention (e.g., refs on DNA/cationic lipids, polymers). After administration the complex is taken up by cells through an endocytotic mechanism, typically involving particle encapsulation in endosomal bodies. The ability of the antisense agent to resist cellular nucleases promotes survival and ultimate delivery of the agent to the cell cytoplasm.

Finally, the ability of the compound to be taken up by cells and form stable heteroduplexes with target RNAs can be tested directly in vivo. Here a labeled test oligomer compound, e.g., fluorescent-labeled compound, targeted against a known mammalian mRNA, e.g., a P₄₅₀ coding sequence, is injected into an animal, e.g., a rat or mouse. 8-24 hours after compound administration, the urine is assayed for the presence of duplex, following the procedures given in Example 1 and 2. If heteroduplex is detected, the compound is suitable for use in the method.

B4. mRNA resistance to RNases. Two general mechanisms have been proposed to account for inhibition of expression by antisense oligonucleotides. (See e.g., Agrawal, et al., 1990; Bonham, et al., 1995; and Boudvillain, et al., 1997). In the first, a heteroduplex formed between the oligonucleotide and mRNA is a substrate for RNaseH, leading to cleavage of the mRNA. Oligonucleotides belonging, or proposed to belong, to this class include phosphorothioates, phosphotriesters, and phosphodiesters (unmodified “natural” oligonucleotides). However, because such compounds would expose mRNA in an oligomer:RNA duplex structure to hydrolysis by RNaseH, and therefore loss of duplex, they are suboptimal for use in the presence invention.

A second class of oligonucleotide analogs, termed “steric blockers” or, alternatively, “RNaseH inactive” or “RNaseH resistant”, have not been observed to act as a substrate for RNaseH, and are believed to act by sterically blocking target RNA nucleocytoplasmic transport, splicing or translation. This class includes methylphosphonates (Toulme, et al., 1996), morpholino oligonucleotides, peptide nucleic acids (PNA's), 2′-O-allyl or 2′-O-alkyl modified oligonucleotides (Bonham, 1995), and N3′ P5′ phosphoramidates (Gee, 1998, Ding).

A test oligomer can be assayed for its ability to protect mRNA against RNaseH by first forming an oligomer: RNA duplex with the test compound, then incubating the duplex with RNaseH under a standard assay conditions, as described in Stein et al. After exposure to RNaseH, the presence or absence of intact duplex can be monitored by gel electrophoresis or mass spec analysis, as described in Examples 1 and 2.

IIC. Uncharged Oligomer Compounds

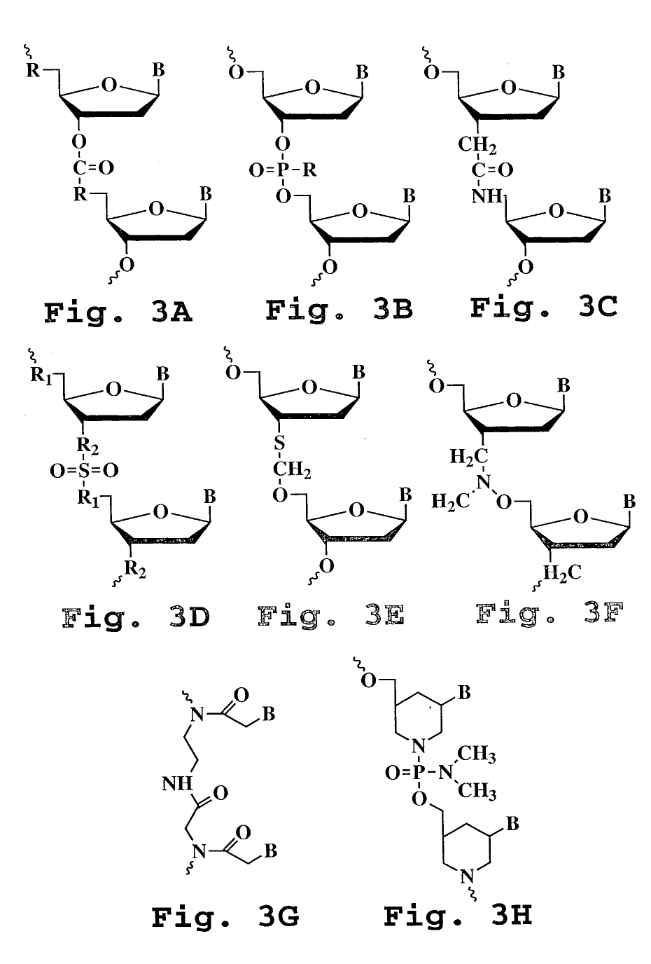

Examples of nonionic linkages in oligonucleotide analogs are shown in FIGS. 3A-3H, and include carbonate (3A, R=O) and carbamate (3A, R═NH₂) linkages, (Mertes, Gait); alkyl phosphonate linkages (3B, R=alkyl or —O-alkyl) (Miller, Jaworska); amide linkage (3C) (Bloomers); sulfone and sulfonamide linkages (3D) (Roughten, McElroy, Egli); and a thioformacetyl linkage (3E) (Cross). The later is reported to have enhanced duplex and triplex stability with respect to phosphorothioate antisense compounds (Cross). Also reported are the 3′-methylene-N-methylhydroxyamino compounds of structure 3F (Mohan).

Pans (FIG. 3G) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm et al., 1993). The backbone of PNAs are formed by peptide bonds rather than phosphodiester bonds, making them well-suited for antisense applications. The backbone is uncharged, resulting in PNA/DNA or PNA/RNA duplexes which exhibit greater than normal thermal stability. PNAs are not recognized by nucleases or proteases. However, PNA antisense agents has been observed to display slow membrane penetration in cell cultures, possibly due to poor uptake (transport) into cells. (See, e.g., Ardhammar M et al., 1999).

One preferred oligomer structure, detailed below, is an uncharged morpholino oligomers such as illustrated by the phosphorodiamidate compound in 3H. Morpholino oligonucleotides (including antisense oligomers) are detailed, for example, in co-owned U.S. Pat. Nos. 5,698,685, 5,217,866, 5,142,047, 5,034,506, 5,166,315, 5,185,444, 5,521,063, and 5,506,337, all of which are expressly incorporated by reference herein.

In testing an oligomer for suitability in the present invention, each of the properties detailed above should be met (recognizing that the “substantially uncharged” feature is inherently met where the linkages are uncharged, and the target-sequence complementarity is achieved by base-sequence design). Thus, the compound should be tested as to its (i) Tm with respect to target RNA at a duplex length preferably between 12-20 basepairs, (ii) ability to be transported across cell membranes by active or facilitated transport, and (iii) ability to prevent RNA proteolysis by RNaseH in duplex form.

C1. Exemplary morpholino compounds. Exemplary backbone structures for antisense oligonucleotides of the invention include the P-morpholino subunit types shown in FIGS. 4A-4E, each linked by an uncharged, phosphorous-containing subunit linkage. Subunit A in FIG. 4 has a phosphorous-containing linkage which forms the five atom repeating-unit backbone shown at A in FIG. 5, where the morpholino rings are linked by a 1-atom phosphoamide linkage.

Subunit B in FIG. 4 is designed for 6-atom repeating-unit backbones, as shown at B in FIG. 5. In structure B, the atom Y linking the 5′ morpholino carbon to the phosphorous group may be sulfur, nitrogen, carbon or, preferably, oxygen. The X moiety pendant from the phosphorous may be any of the following: fluorine; an alkyl or substituted alkyl; an alkoxy or substituted alkoxy; a thioalkoxy or substituted thioalkoxy; or, an unsubstituted, monosubstituted, or disubstituted nitrogen, including cyclic structures.

Subunits C-E in FIG. 4 are designed for 7-atom unit-length backbones as shown for C through D/E in FIG. 5. In Structure C, the X moiety is as in Structure B and the moiety Y may be a methylene, sulfur, or preferably oxygen. In Structure D the X and Y moieties are as in Structure B. In Structure E, X is as in Structure B and Y is O, S, or NR. In all subunits depicted in FIGS. 3A-E, Z is O or S, and Pᵢ or Pᵢ is adenine, cytosine, guanine or uracil.

One preferred “morpholino” oligonucleotide is composed of morpholino subunit structures of the form shown in FIG. 5B, where (i) the structures are linked together by phosphorodiamidate containing linkages, one to three atoms long, joining the morpholino nitrogen of one subunit to the 5′ exocyclic carbon of an adjacent subunit, (ii) Pᵢ and Pⱼ are purine or pyrimidine base-pairing moieties effective to bind, by base-specific hydrogen bonding, to a base in a polynucleotide, and X═NH₂, Y═O, and Z═O.

The important chemical properties of a morpholino-based subunit are the ability to be linked in a polymeric form by stable, uncharged backbone linkages, the ability of the polymer so formed to hybridize with a complementary-base target nucleic acid, including target RNA, with high Tm, even with oligomers as short as 10-14 bases, the ability of the oligomer to be actively transported into mammalian cells, and the ability of the oligomer:RNA heteroduplex to resist RNAse degradation.

C2. Oligomer synthesis and modifications. The antisense compounds of the invention can be synthesized by stepwise solid-phase synthesis, employing methods detailed in the references cited above. The sequence of subunit additions will be determined by the selected base sequence (see Section D) below.

In some cases, it may be desirable to add additional chemical moieties to the oligomer compounds, to enhance the pharmacokinetics of the compound or to facilitate capture or detection of a heteroduplex containing the compound. The moiety is covalently attached typically to the 5′- or 3-end of the oligomer according to standard synthesis methods.

For example, addition of a polyethyleneglycol moiety or other hydrophilic polymer, e.g., one having 10-100 polymer subunits, may be useful in enhancing the solubility of an oligomer compound.

One or more charged groups, e.g., anionic charged groups such as an organic acid, may enhance cell uptake.

A reporter moiety, such as fluorescein or a radiolabeled group, may be attached for purposes of detecting the presence of heteroduplex in the body sample. Alternatively, the reporter label attached to the oligomer may be ligand, such as an antigen or biotin, capable of binding a labeled antibody or streptavidin.

Finally, the oligomer may be provided with a sequence-associated antigen, such as 2-4 dinitrophenol and related antigens, through which a heteroduplex containing that sequence can be captured specifically with an antigen-specific antibody.

In selecting a moiety for attachment or modification of an oligomer antisense, it is generally of course desirable to select chemical compounds of groups that are biocompatible and likely to be tolerated by the subject without undesirable side effects.

IID. Targets For Antisense Oligonucleotides

This section considers the five classes of target RNA's mentioned above, and provides exemplary sequences from which target sequences can be selected. As a rule, sequences for genes or microorganisms of interest, such as those specifically mentioned below, may be found in public gene databases, such as the NCBI GenBank database (www.ncbi.nlm.nih.gov/GenBank).

Where the target sequence may be any sequence within a given RNA, e.g., processed mRNA or viral genome, the selection of a target sequence is generally made by selecting a sequence at least 12-15 bases in length (to optimize sequence uniqueness) and within that size range, a sequence that that is rich in C:G basepairs, for higher Tm values. It is generally not important or even desirable to select a target sequence that is critical to mRNA processing or translation, such as an AUG start site or splice junction site, since the administered antisense would then have a potentially disruptive effect of cell metabolism. However, the method may be carried out as part of a therapeutic antisense treatment method, where the antisense agent administered is designed to disrupt RNA processing or translation, and the same agent is used, in accordance with the present invention, to monitor or confirm antisense delivery to the target RNA.

Where the antisense agent is designed to bind to an RNA having a single base-pair mutation, one of three strategies for discriminating mutated from wildtype sequences can be followed. The first relies on the potential of intracellular RNAse to cleave heteroduplex RNA at a base mismatch in the heteroduplex. For example, if the antisense compound is 12-14 bases in length, and contains the base complementary to the mutated base at a central position, RNAse cleavage of the heteroduplex (which would occur with the wildtype, but not the mutated sequence) would be effective to denature the heteroduplex. Thus, heteroduplex would be detected only if the mutation-containing target sequence were present.

In a second strategy, the antisense compound contains, in addition to a base complementary to the mutation-site base, a mispaired base close to the mutation site, e.g., 2-5 bases from the mutation-base site. For example, in a 14mer agent, the base-site mutation may be a position 6, and the mispair at position 11. This compound is designed to form a heteroduplex that permits a single-base mispair at physiological temperature (the mutated target sequence), but is unable to form a stable heteroduplex with sequences containing two mispairs (the wildtype target sequence). Even if the mispaired base in the heteroduplex becomes a site if RNAse cleavage, the heteroduplex will still be stable by virtue of an 8 or greater contiguous base pairing in the heteroduplex.

A third strategy is based on competition for target binding between two antisense agents: one having a sequence complementary to the mutated target and carrying one reporter, e.g., a first fluorescent reporter, and a second agent having a sequence complementary to the analogous wildtype target sequence, and carrying a distinguishable second reporter, e.g., a second, fluorescent reporter. In cells expressing the mutated sequence, the ratio of heteroduplex formed with the first agent to those formed with the second agent will be high, and correspondingly low where only the wildtype sequence is expressed.

D1. Genes whose expression is to be inhibited by a therapeutic antisense. A large number of genes are potential therapeutic targets for antisense therapy. In general, the rationale of the antisense therapy is to disrupt processing or translation of the gene transcript (mRNA), thus inhibiting expression of the target gene. Among the large number of genes that have been proposed as targets for antisense therapy are (along with the corresponding GenBank sequence number) the following: methionine aminopeptidase 2 (NM_{—}006838); Interleukin-5 (J03478); C-myc (X00364); C-myb (M15024); PI3 kinase p110 (S67334); focal adhesion kinase (L13616); telomeric repeat binding factor 1 (NM_{—}017489); PDK-1 (L42450); intercellular adhesion molecule-1 (X84737); G-alpha-S1 (X04409); SRA (AF092038); G-alpha-16 (M63904); P13 kinase p85 (AC007192); MEK1 (L11284); RAF (X03484); thymidylate synthase (D00596); 17. X-linked inhibitor of apoptosis (U45880); TNF-alpha (M16441); MEKK5 (AL024508); survivin (U75285); MDMX (AF007111); liver glycogen phosphorylase (AF046787); SMAD5 (AF010602); SMAD2 (AF027964); PEPCK-mitochondrial (NM_{—}004563); RhoC (L25081); PTEN (AH007803); RIP-1 (U55766); FADD (NM_{—}003824); SMAD3 (SEG_AB004922S); EGR-1 (AJ243425); TNFR1 (AH003016); mcl-1 (AF118124); microtubule-associated protein 4 (NM_{—}002375); sentrin (U83117); interleukin-15 (U14407); B-RAF (M95712); integrin alpha 4 (L12002); her-2 (AF177761); RhoG (NM_{—}0016655); RhoB (X06820); MEK2 (L11285); serine/threonine protein phosphatase (X97867); ELK-1 (Y11432); RhoA (L25080); bcl-xl (Z23115); Atm (SEG_D83244S); DIR1 (AF139374); Bcl-2 (U16812); mdr1 (AF016535); polo-like kinasel (X73458); protein kinase C-alpha (NM_{—}002737); TGF-alpha (M31172); telomerase (AF047386); amphiregulin (M30704); TNF-alpha (X02910, X02159); IGF-1 (A29117); TGF-beta (M60316); TR3 orphan receptor (L13740); topoisomerase 11 (J04088); bcr/abl (AJ131467 partial); urokinase (E00178); connexin43 (U64573); p53 (AH002918); basic fibroblast growth factor (J04513); c-kit protooncogene (L04143); ETS-2 (J04102); NF-kappa-B p65 (L19067).

D2. Genes whose expression indicates a given biochemical state. Certain conditions, or predisposition to certain conditions, are characterized by the altered expression of RNAs or RNA translation products (i.e. peptides or proteins) which are not expressed in normal cells. Typically, the gene products, i.e., proteins, are detected in the patient's blood, and used to diagnose a particular condition or propensity toward a particular condition. In particular, gene proteins have been identified that are diagnostic of (i) predisposition to various cancers, (ii) prognosis or treatment response in cancer patients, (iii) predisposition to alcoholism, (iv) predisposition to heart disease, (v) liver pathologies, and (vi) neurological pathologies, e.g., Alzheimer's disease. Below is a partial list of genes that have been identified in each of these six classes, along with the corresponding GenBank sequence numbers.

D2(i). Predisposition to various cancers: 5. fes (NM_{—}002005); fos (K00650); myc; myb; fms (U63963); multi-drug resistance-associated protein (MRP) (L05628); lung resistance protein (LRP); p53 gene (AH007667); retinoblastoma gene (L11910); Wilm's tumor gene (M64241); and human mismatch repair gene hMSH2 (AH003235).

D2(ii) prognosis or treatment response in cancer patients: pro-gastrin-releasing peptide (AH002713); SCC antigen (SCC-Ag) (S66896); UPA (X02419); PAI-1 (AH002922); HER-2 (AF177761); vascular-endothelial-growth-factor (VEGF) (M32977); insulin-like growth factor I (M37484, M29644); IFG-binding protein 3 (M35878); bcl-2 (U16812); HER-2/neu oncogene (AH002823); cytokeratin 20 (X73501); sex hormone binding globulin (M31651); IL-2 receptor (E00727); alpha-fetoprotein (NM_{—}001134); interferon-inducible MxA protein (NM_{—}002462); TNF-b (D12614); fatty acid synthase (OA-519) (NM_{—}004104); tetranectin (X98121); C-erbB-2 (AH001455); P-glycoprotein (M14758); carcinoembryonic antigen (M17303); chromogranin A (AH005196); Haptoglobin-related protein (Hpr) (NM_{—}020995); Pregnancy-associated plasma protein A (NM_{—}002581); alkaline phosphatase (J04948).

D2(iii) predisposition to alcoholism: gamma-glutamyl transferase (J04131); gamma-glutamyl transpeptidase (J04131); D2 dopamine receptor (M29066); CYP1Al (NM_{—}000499); alpha-1-antitrypsin P1 (K01396, Ml 1465); haptoglobin HP (M69197); alcohol dehydrogenase (ADH) (X76342); aldehyde dehydrogenase (ALDH) (AH002598);

D2(iv) predisposition to heart disease: lipoprotein-associated phospholipase A2 (U24577); adrenomedullin (NM_{—}001124); C-reactive protein (M11880).

D2(v) liver pathologies: alpha-L-fucosidase (AH002702); gastrin (AH005301).

D2(vi) neurological pathologies, e.g., Alzheimer's disease: presenilin 2 (D84149 partial); acetylcholinesterase (M55040); beta 2-microglobulin (AF072097); apolipoproteins E (K00396).

D3. Genetic mutations. A large number of genetic mutations associated with genetic diseases, or the predisposition to genetic diseases have been identified. See, for example, Schroeder, H. W., cited above, which is incorporated herein by reference. For example, Table 23-3 of the Schroeder reference lists the most common genetic disorders grouped by autosomal dominant, autosomal recessive, and X-linked; Table 24-3, which lists diseases caused by mutations in plasma membrane protein(s); Table 244, which lists disorders caused by various identified mutations in the human glucose transporter; and Table 24-5, which lists a number of mutations in the human insulin receptor gene. One skilled in the art could readily determine from these and other references widely available, particular mutations associated with a large number of genetic disorders, including the GenBank sequence resource, and design oligomers to target the mutated sequences, following the principles outlined above for discriminating between wildtype and single-mutation sequences.

D4. Viral Genomic Sequences.

The methods of the invention find further utility in monitoring the infection of a subject by any of a number of microorganisms and the effect of therapeutic intervention on such infection. More specifically, infection with particular viruses, bacteria or fungi may be diagnosed and therapy monitored by evaluating the expression of RNA or DNA associated with such infection using the methods of the invention. Characteristic nucleic acid sequences which are associated with a large number of infectious microorganisms are available in public databases and may serve as the basis for the design of specific antisense oligomers for use in the methods of the invention.

For example, typically viral infections (e.g., those caused by the expression of a latent virus such as CMV) are monitored by analysis of infected tissue or blood using immunofluorescence assays, polymerase chain reaction (PCR), and/or enzyme-linked immunosorbent assay (ELISA). The presence of a virus in a broad class of viruses such as Retroviridae, Papovaviridae, Herpesviridae, and Paramyxoviridae can be determined. The presence of specific viruses within these classes, such as T cell leukemia-associated viruses (HTLV-1, HTLV-II), Human immunodeficiency virus (HIV) 1 and 2, sarcoma and leukemia viruses, Simian virus 40 (SV40), herpes simplex type 1 and 2, Epstein-Barr virus, parainfluenza viruses, mumps virus, and measles virus can further be determined. The sequences of target viruses can be obtained from Genbank.

More specifically, a general embodiment for use in identifying the viral infective agent in an infected subject includes first and second oligomer compositions. The first composition includes oligomers that target broad families and/or genera of viruses, e.g., Retroviridae, Papovaviridae, Herpesviridae, and Paramyxoviridae. Oligomers in this composition can be determined from standard GenBank viral sequences, where the desired sequences are viral sequences (i) specific to broad virus family/genus, and (ii) not found in humans. The second composition includes oligomers complementary to specific genera and/or species and/or strains within a broad family/genus. Several different second oligomer compositions—one for each broad virus family/genus tested in the first composition are required. For the second compositions, sequences are selected which are (i) specific for the individual genus/species/strains being tested and (ii) not found in humans.

D5. Bacterial and Fungal Sequences.

The method of the invention is further applicable to detecting bacterial or fungal infective agents, and for obtaining information useful in treatment, e.g., whether the infective bacteria is drug resistant, and if so, the type of drug-resistance genes.

In a preferred embodiment, the method utilizes two oligomers compositions, analogous to those used for detecting an infective viral agent. A first composition includes a plurality of oligomer sequences targeted to broad families and/or genera of bacteria or fungal organisms, e.g., the families of bacteria given below. For each broad bacterial family/genus targeted, the oligomer composition contains an oligomer targeted against a bacterial sequence that is (i) specific to the broad family/genus or bacteria, and (ii) not found in humans. Broad family or genus-specific sequences are known, for example for bacterial 16S and 23S rRNA that represent useful targets, such as detailed in co-owned U.S. patent application for “Antibacterial Method and Composition, filed Nov. 29, 2000, which is incorporated herein by reference.

For each oligomer in the first composition, a second composition provides a plurality of oligomers directed against specific genera/species/or strains in the broad family/genus group. Some common pathogenic bacterial species and GenBank sequences associated with them are as follows: Escherichia coli (X80725); Salmonella thyphimurium (U88545); Pseudomonas aeruginosa (AF170358); Vibrio cholera (AF118021); Neisseria gonorrhoea (X07714); Staphylococcus aureus (Y15856); Mycobacterium tuberculosis (X52917); Helicobacter pylori (M88157); Streptococcus pneumoniae (AF003930); Treponema palladium (AJ010951); Chlamydia trachomatis (D85722); Bartonella henselae (X89208); Hemophilis influenza (M35019); Shigella dysenterae (X96966).

Another useful target are sequences directed to bacterial drug-resistance genes, allowing the treating physician to identify the infecting organism, and to choose the most favorable antibiotic for treatment, based on the drug-resistance profile of the infecting organism.

III. Modes of Practicing the Invention

In practicing the method of the invention, an antisense compound or alternatively, or composition containing a plurality of different-sequence antisense compounds is administered to a subject, e.g., a human subject. If the purpose of the method is to detect the presence of one or more genetic mutations, the compound or composition may be administered once only at any convenient time.

If the purpose of the method is to detect up- or down-regulation of a selected gene of genes in response to a given condition or therapeutic treatment, the compound or composition is given at a selected time or times before and/or after the condition or treatment. For example, to monitor the effect of a drug to up-regulate a given gene, a compound targeted to the gene's mRNA is administered before administration of the drug, to establish a “control” level of the mRNA, then again at a selected interval, e.g., 4-24 hours, after drug administration, to determine mRNA level in response to the drug.

Following administration of the antisense compound or composition (multiple antisense compounds) to the subject, the compound(s) are allowed to biodistribute within the subject as outline in the model shown in FIG. 1. At one or more selected time intervals following administration, a body-fluid sample is taken, and the presence and/or amounts of one or more heteroduplex species in the sample is determined/measured. The sampling times are typically in the range 4-24 hours post administration, preferably 8-16 hours, although a series of samples, e.g., every four-eight hours for up to 24 hours post administration may be suitable.

The body sample is then assayed to determine the presence and/or amount of heteroduplex or different heteroduplexes in the sample. The following subsections consider detailed methods and devices for carrying out the methods.

IIIA. Administering Antisense Oligomers

Effective delivery of the oligomer compound may be accomplished by any of a number of methods known to those of skill. Such include, but are not limited to, oral delivery, various systemic routes, including parenteral routes, e.g., intravenous, subcutaneous, intraperitoneal, intramuscular, and intra-arterial injection, as well as inhalation and transdermal delivery. In some cases targeted delivery by direct administration to a particular tissue or site is preferred. It is appreciated that any methods that are effective to deliver the drug to a target site or to introduce the drug into the bloodstream are also contemplated.

Targeting of antisense oligomers may also be accomplished by direct injection into a particular tissue or location, i.e., direct injection into a tumor, thereby facilitating an evaluation of expression of a particular RNA sequence associated with the tumor (i.e. a tumor suppressor gene or an oncogene). Alternatively, the antisense oligomer may be conjugated with-a molecule which serves to target the oligomer to particular tissue or cell type, e.g., an antibody/oligomer conjugate.

Transdermal delivery of antisense oligomers may be accomplished by use of a pharmaceutically acceptable carrier adapted for e.g., topical administration. One delivery vehicle, discussed further below, includes a solution of 50-90% ethylene glycol in aqueous medium, and an antisense compound at an amount of between 0.05 to 3 mgs, in an area of 1 cm².

Preferred doses for oral administration are from about 1 mg oligomer/patient to about 25 mg oligomer/patient (based on a weight of 70 kg). In some cases, doses of greater than 25 mg oligomer/patient may be necessary. For IV administration, the preferred doses are from about 0.5 mg oligomer/patient to about 10 mg oligomer/patient (based on an adult weight of 70 kg).

The antisense compound is generally administered in an amount and manner effective to result in a peak blood concentration of at least 200400 nM antisense oligomer. The presence of heteroduplex in a body fluid, e.g., urine is monitored typically 3-24 hours after administration, preferably about 6-24 hours after administration.

IIIB. Sample Collection and Treatment

At selected time(s) after antisense administration, a body fluid is collected for detecting the presence and/or measuring the level of heteroduplex species in the sample. As indicated above, the body fluid sample may be urine, saliva, plasma, blood, spinal fluid, or other liquid sample of biological origin, and may refer include cells or cell fragments suspended therein, or the liquid medium and its solutes. The amount of sample collected is typically in the 0.1 to 10 ml range. preferably about 1 ml of less. Where the sample is obtained from a skin region (see below), the sample “volume” is an amount of skin removed by an adhesive from a skin region having an area typically between 1-25 mm².

The sample may be treated to remove unwanted components and/or to treat the heteroduplex species in the sample to remove unwanted ssRNA overhang regions. Example 1 describes sample treatment with RNase to remove any single-stranded RNA overhand in the heteroduplex. It is, of course, particularly important to remove overhang where heteroduplex detection relies on size separation, e.g., electrophoresis of mass spectroscopy.

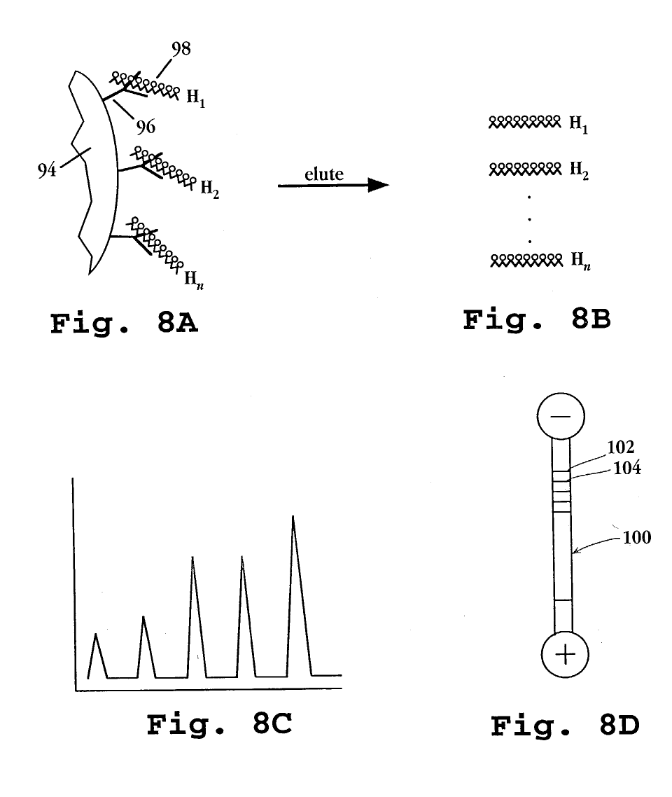

A variety of methods are available for removing unwanted components. Fort example, since the heteroduplex has a net negative charge, electrophoretic or ion exchange techniques can be used to separate the heteroduplex from neutral or positively charged material. A more specific technique, which is described further below with respect to FIG. 8, is to contact the sample with a solid support having a surface-bound antibody or other agent specifically able to bind the heteroduplex. After washing the support to remove unbound material, the heteroduplex can be released in substantially purified form for further analysis, e.g., by electrophoresis of mass spectroscopy, as described below.

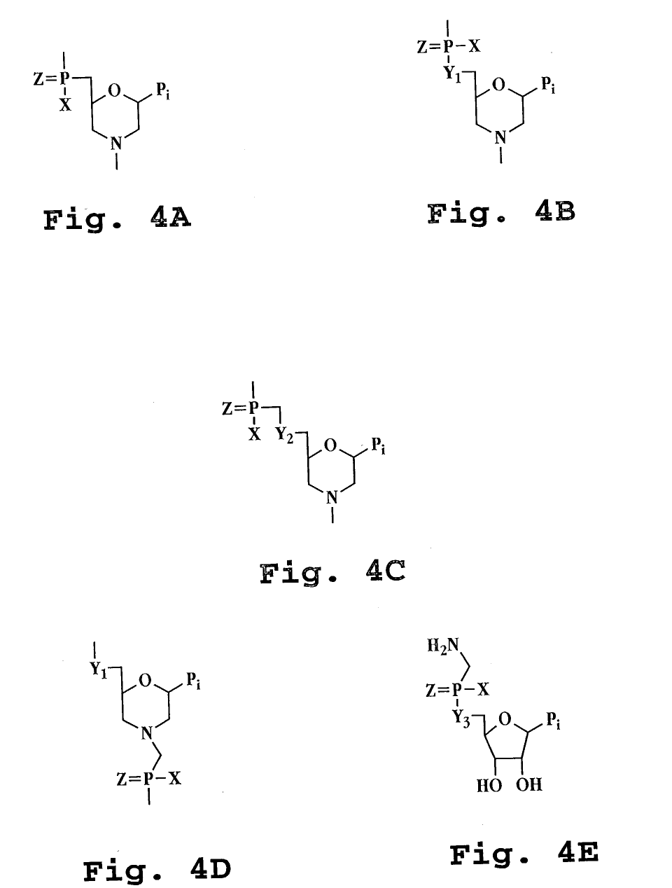

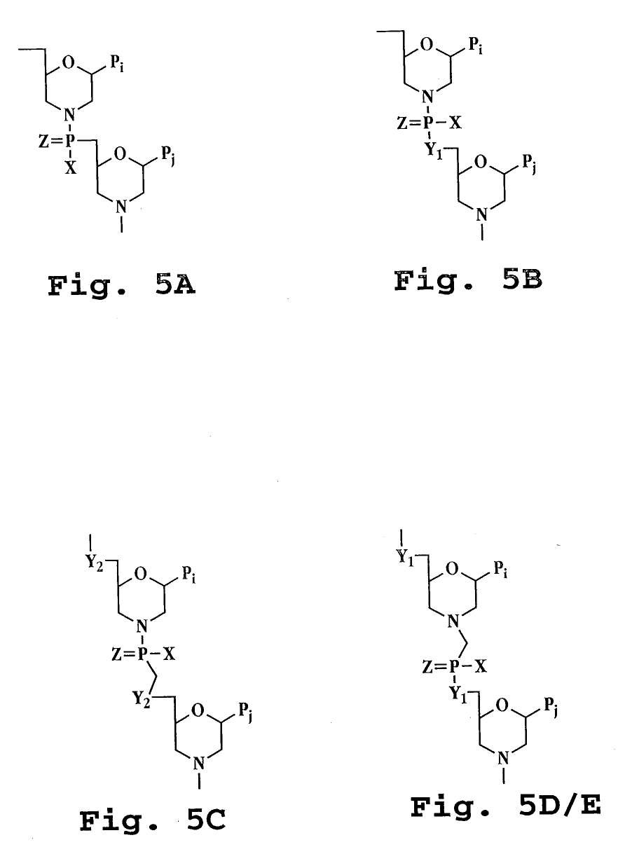

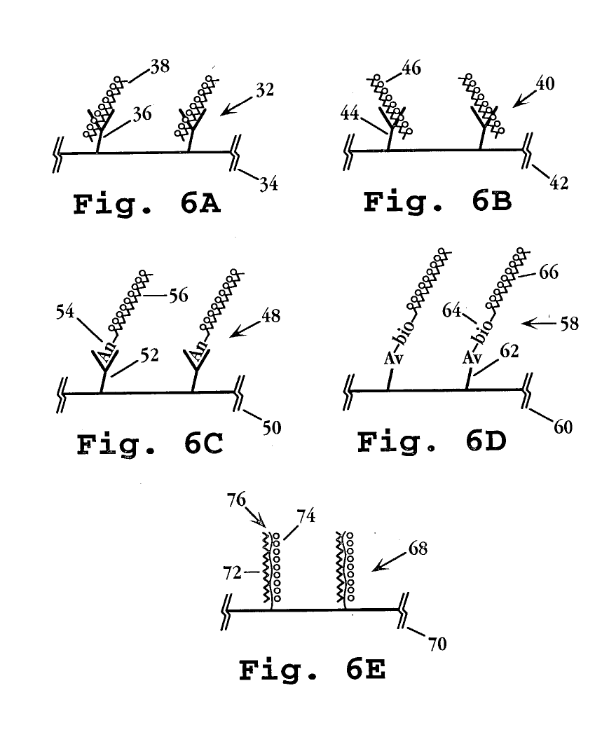

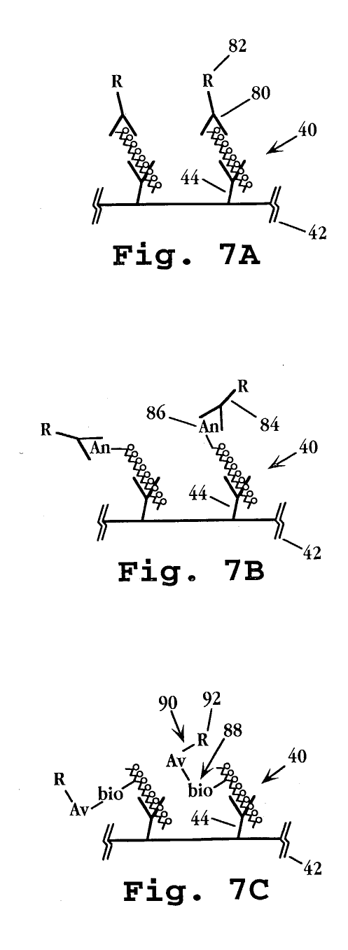

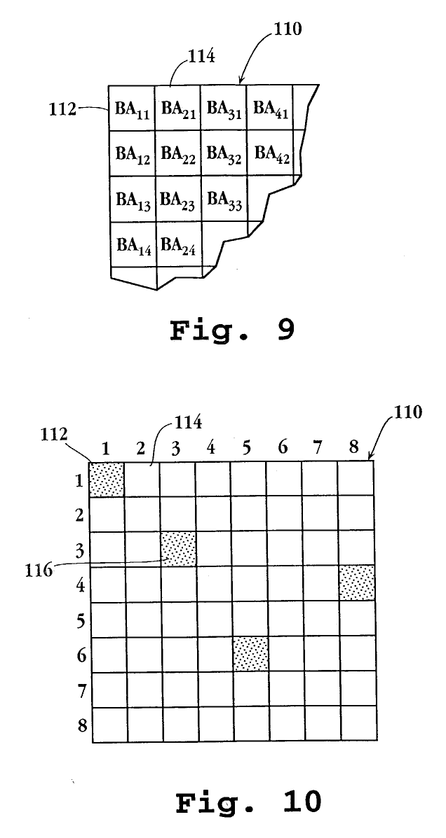

Alternatively, the detection/measuring step can be carried out on a solid support having a surface-bound antibody or other agent capable to reacting specifically with a heteroduplex or the antisense component thereof, as described below with respect to FIGS. 6 and 7. In this embodiment, the sample is brought into contact with the solid support, under heteroduplex binding conditions. After washing the support to remove unbound material, the support is further reacted with reporter reagents designed to bind to support-bound heteroduplex. This approach is particular advantageous in an array format for detecting a plurality of different-sequence heteroduplex species, as detailed below with reference to FIGS. 9 and 10.

IIIC. Detecting of Heteroduplex

Heteroduplex present is a body sample, such as urine, saliva, blood, hair, or a skin-cell sample, may be assayed by solid-phase or fluid-phase assay methods. In general, a solid-phase reaction involves first binding heteroduplex analyte to a solid-phase support, e.g., particles or a polymer or test-strip substrate, and detecting the presence/amount of heteroduplex bound to the support. In a fluid-phase assay, the analyte sample is typically pretreated to remove interfering sample components, then analyzed in solution or gas-suspension form, e.g., mass spectroscopy.

C1. Solid-phase format. FIGS. 6A-6E illustrate various heteroduplex binding agents useful in the present invention for a solid-phase format. FIG. 6A shows a device 32 having a substrate or support at 34 and a surface bound heteroduplex binding agent 36. In this embodiment, the binding agent is an antibody whose binding affinity is specific for a heteroduplex formed of target RNA and a known oligomer, but is non-specific as to heteroduplex base sequence.

The antibody, which forms one aspect of the invention, is formed by standard methods, such as outlined in Example 3. Briefly, a selected oligomer agent, such as PMO having a 3′ triethyleneglycol tail, is coupled at one or its ends to a suitable carrier, such as keyhole limpet hemocyanin (KLH) by standard linker chemistry. The oligomer-carrier KLH conjugate is hybridized to complementary RNA, then injection into mice, followed by boosting and bleeding the mice to determine whether strong antibody titer to PMO existed. Hybridoma cells lines are produced by immortalizing spleen cells from the immunized animals, according to standard hybridoma technology.

Monoclonal antibodies (Mabs) from various hybridoma cell lines are then tested for specificity to the antigen. Among the antibodies identified in Example 3 were those (i) specific against the heterodimer, but non-specific as to heteroduplex base sequence, (ii) specific against both heteroduplex and heteroduplex sequence, and (iii) specific against the triethylene glycol tail of the heterodimer. It will be appreciated how the method provided in Example 3 can be applied to heterodimers formed with any selected oligomer compounds.

Antibodies formed as above are attached to the substrate surface by well known protein attachment methods, such as covalent coupling to a substrate reactive groups using a bivalent coupling agent, via ester, amide, thioether, disulfide, or other linkages. U.S. Pat. Nos. 5,516,635 and 5,837,551 are representative teachings disclosing antibody coupling to a solid-support.

FIG. 6B shows a similar type of solid-phase device 40 having a substrate 42, and a surface-attached binding agent 44 capable of sequence-specific binding to a selected heterodimer. That is, the antibody shows high affinity binding only for a particular oligomer:RNA heterodimer structure having a particular duplex base sequence. Methods for producing such antibodies, which form an aspect of the invention, are as discussed above and in Example 3.

FIG. 6C, device 48 has a substrate 50 and surface-bound antibodies, such as antibody 52, that shows high-affinity binding for an antigen 54 coupled the oligomer compound. The antigen may be, for example, an amino acid or oligopeptide or a small molecular-weight antigen, such as dinitrophenol or oliogethyleneglycol. The antigen is attached to one end, e.g., the 3′-end of the oligomer moiety of the heteroduplex, using conventional coupling methods. Antibodies against the antigen may be formed against the antigen alone, e.g., coupled to KLH, or the antigen in combination with the oligomer compound. Example 3 below discloses the production of a Mab against the triethylene-glycol moiety of a derivatized PMO agent. The antibody is coupled to the substrate surface by conventional methods as above.

Device 58 in FIG. D is similar, except that the ligand attached to the oligomer agent the heterodimer 66 is a biotin group 64, and the antiligand binding agent 62 attached to substrate 60 is avidin. In this embodiment, the oligomer compound is synthesized with one or more biotinylated bases, according to known methods, and avidin is attached to the substrate surface also by well-known methods.

Device 68 in FIG. 6E has a substrate 70 with surface bound oligomer binding agents 72 designed to bind in a sequence specific manner with an RNA oligomer heteroduplex, by forming a base-specific triple helix with the heteroduplex. Oligomers capable of forming triple-stranded helical structures with oligonucleotide or oligonucleotide-analog duplexes are detailed, for example in U.S. Pat. No. 5,844,110, which discloses nucleotide-analog oligomers having quinoline- or quinozoline-based structures capable of hydrogen bonding specifically with interstrand purine-pyrimidine base pairs in a double-stranded Watson-Crick DNA structure. Although the oligomer structures disclosed in the patent have phosphodiester-linked ribose or deoxyribose backbone structures, there is no absolute requirement for charged ribose-based backbones, since the polymer backbone is functioning only to place the modified bases at positions capable to binding to major groove sites in the duplex. Thus, any regular polymer backbone capable to carrying the modified bases at desired spacing corresponding to the base-to-base spacing of the duplex structure should be suitable.

Another duplex-binding agent capable of forming stable base-specific triple strand structures with duplex nucleic acids is disclosed in co-owned U.S. Pat. Nos. 5,405,938 and 5,166,315, both of which disclose polymer compositions having uncharged 5- or 6-membered cyclic backbones, e.g., uncharged ribose or morpholino, and modified bases designed to bind hydrogen bond specifically with different oriented basepairs in target duplex structures.

The duplex binding oligomer is attached to a solid support by conventional surface-attachments chemistries, such as those cited above. In the case where the surface-attached oligomers are prepared by subunit addition in solid-phase, the solid phase on which the particles are prepared may be the device substrate or support itself.

In performing a detection assay, the sample in solution is placed in contact with the support surface, and allowed to react under conditions that allow analyte binding to binding-agent molecules. Typically, the binding reaction is carried out at physiological pH, at a temperature between 24-37° C., for a reaction time of 5-30 minutes, depending on the particular ligand-anti-ligand binding reaction. At the end of the reaction period, the substrate may be washed one or more times with buffer and/or mild detergents to remove non-specifically bound sample material.

FIGS. 7A-7C illustrate various methods by which heteroduplex can be detected in a solid-phase format. The device shown here, which is representative, is device 40 in FIG. 6B, having as binding agents 42, antibodies that bind specifically to heteroduplex independent of heteroduplex sequence. In the embodiment shown in FIG. 7A, the detection agent is an antibody 80 specific against the heteroduplex, as above, carrying a reporter group 82, such as a fluorescent moiety, gold particle, chromophore, or other detectable reporter group. Methods for forming antibody/reporter conjugates are well known.

FIG. 7B illustrate a similar antibody detection agent 84, but where the antibody is immunospecific against an antigen 86 carried on the oligomer compound in the heteroduplex, as discussed above with respect to FIG. 6C.

In the detection format illustrated in FIG. 7C, the oligomer in the heteroduplex contains one of more biotinylated bases, indicated at 88, as described above with respect to FIG. 6D, and the detection agent 90 includes avidin conjugated to a reporter group 92.

It will be appreciated that other detection agents will be suitable for use in detecting a support-bound heteroduplex. For example, in the embodiments shown in FIGS. 6C and 6D, where the heteroduplex is bound to the solid support through a 3′- or 5′-end ligand, the binding agent may be a reporter-labeled triple-strand oligomer of the type described above, or reporter-labeled cationic polymer, such as polyethylamine, which is able to bind to heteroduplex by charge interactions with the charged RNA backbone of the heteroduplex.

The detection agents above are designed direct-binding assays where heteroduplex is initially reacted with the solid support in the absence of any competing heteroduplex species. The invention also contemplates competitive assays in which sample reacted with the solid support in the presence of a known amount/concentration of reporter-labeled heteroduplex, where the amount of labeled heteroduplex bound to the solid support may be inversely related to the amount of heteroduplex contained in the assay sample. Labeled heteroduplex can be prepared by labeling either the oligomer or RNA strands of the heteroduplex.

A competitive assay format may be designed conventionally as a test strip to include the competing, labeled heteroduplex in the flow path of the sample, such that sample flow through the test strip is effective to simultaneously bring sample and labeled heteroduplex to a region of heteroduplex binding on the strip. It will be appreciated that the present method can be adapted a variety of other known solid-phase two-step or homogenous assays involving ligand/anti-ligand interactions.

The presence and/or amount of bound reporter can be measured/determined by conventional methods, which may involve visual inspection, or quantitative detection by a machine reader, e.g., a standard colorometric or fluorometric card, slide or array reader.

The device and detection reagent for use in detecting a selected oligomer:RNA heteroduplex form a kit, in accordance with another aspect of the invention, which may also include the oligomer compound in a suitable delivery form. For example, a home pregnancy test kit, in accordance with this aspect of the invention, might include a PMO oligomer having an hCG-specific sequence, e.g., the sequence above, in tablet form for oral delivery, and a solid-phase test strip having free (mobile) labeled anti-heteroduplex antibody contained therein, and a binding-agent detection area on the strip, for a conventional sandwich assay.

In self-testing for pregnancy, the user would ingest the oligomer-containing table, and at a selected later time, e.g., 12-24 hours post administration, collect a urine sample. To detect the presence of telltale hCG-sequence heteroduplex, the test strip is dipped in the urine sample, which is sequence heteroduplex, the test strip is dipped in the urine sample, which is allowed to migrate along the length of the strip where heteroduplex analyte successively binds to (i) free labeled anti-heteroduplex antibody, to label the heteroduplex with a detectable reporter, and (ii) immbolized anti-heteroduplex antibody, to bind the labeled analyte at a sample-detection region. The assay readout, indicating the presence of target hCG, is simply the presence of detectable reporter at the detection site on the strip.

A variety of other test kits, incorporating oligomer sequences corresponding to those indicated in above, and having any of a variety of known assay formats, are also contemplated herein, e.g., for detecting the presence of one or more genetic mutations characterized by a point mutation or a pathological condition characterized by the presence or absence or levels of a given gene product, or for detecting an identifying a given viral, bacterial, or fungal infective agent.

C2. Fluid-phase detection. Fluid-phase formats, broadly connotes detection of heteroduplex in a liquid medium, a simple solution for a homogeneous assay, in a separation medium, e.g., a gel electrophoresis or liquid-chromatographic separation medium, or gas-carrier phase, such as in mass spectrometry. In general, the sample being assayed will have been pretreated to remove interfering substances.

One general method for pretreating a sample is illustrated in FIG. 8A, which shows a solid support 94 having surface bound binding agents 96, such as heteroduplex specific antibodies. Initially, a liquid sample containing heteroduplex analyte is reacted with the solid support under analyte binding conditions, then washed to remove non-specifically bound sample material. Following this, the heteroduplex may be released from the solid support, e.g., by conventional methods, and eluted into a solution or gas carrier for heteroduplex analysis, e.g., by electrophoresis or mass spectroscopy. As will be seen below, these methods are particularly well suited to identifying heteroduplex analytes in a sample mixture containing a plurality of different-sequence heteroduplex analytes.

IV. Multi-Analyte Sample Method

In many cases, it is desirable or necessary to assay a plurality of different RNA targets. For example, when testing an individual for genetic diseases, often a battery of tests for different genetic diseases is carried out, particularly in fetal genetic screening. Similarly, when testing for an infective agent by genetic analysis, it is generally necessary to include sequence probes for a large number of candidate organisms.

In one general embodiment, for use in genetic screening, the invention includes a composition containing a plurality of oligomer compounds whose sequences are targeted to each of a plurality of known genetic mutations, such as those identified above. Where the composition is administered to a pregnant woman, for use is genetic screening of fetal mutations, the compound sequences are targeted against genetic abnormalities commonly tested for by fetal genetic screening, such as Down's syndrome.

In a second embodiment, the composition of the invention includes a plurality of oligomer compounds whose sequences are targeted against mutations in oncogenes or suppressor genes, such as those listed in Section II above, which are associated with cancer or a predisposition to cancer.

In another embodiment, the composition includes a plurality of oligomers whose sequences are targeted against various genes, such as . . . , that are indicative of two or more pathological conditions, or a disposition to pathological conditions, such as diabetes, liver disease, heart disease, and neurological disorders, as given above.

In still another embodiment, for use in detecting and identifying a given infected viral, bacterial, or fungal agent, the composition contains oligomers whose sequences are target against groups or classes of microorganisms. For example, to detect infection by an unknown viral organism, the patient may be given an initial composition containing sequences directed against broad families of viral pathogens. After initial detection and identification of viral family, the patient can be administered a second, more specific group of oligomers, for identification of particular viral species or strains within the first-identified family. Likewise, for detecting a bacterial pathogen, a first composition may be designed for identifying a bacterial family or genus, and a second more compositions, for detecting particular species or strains within the first class.

In a related embodiment, for identifying an optimal treatment method for a patient having a bacterial infection, the composition may include oligomers whose sequences are targeted against known mutations associated with certain types of drug-resistance, to identify no only bacterial pathogen, but the type of antibiotic which is likely to be most effective in treating the infection.

According to an important aspect of the invention, the assay methods and kits described above for non-invasive detection of target RNA sequence are readily adaptable to assay formats in which a plurality of different target sequences are detected and or quantitated. The methods and kits for multiple-analyte analysis generally follow those described above, but with the following differences.

1. The oligomer material administered to the subject contains a plurality, i.e., two or more, different oligomer compounds targeted against a plurality of different sequences, as indicated above. The different sequences may be administered as a composition, e.g., oral tablet or injectable solution, containing multiple oligomer compounds, or as an array, for transdermal delivery, as will be detailed in Section IVC below.

2. Heteroduplex detection requires tools or methods for identifying the individual different-sequence heteroduplexes that are formed and present in the sample. In a general fluid-phase assay format, detailed in Section IVA below, different-sequence heteroduplexes are detected on the basis of different physical-separation properties, allowing the heteroduplexes to be distinguished on the basis of, for example, electrophoretic mobility, mass spectrographic characteristics, or chromatographic properties. In a general solid-phase assay format, detailed in Example IVB, sample material is reacted with an array of sequence-specific duplex binding agents, such that each different-sequence analyte binds to a known-sequence region of the array. A modified solid-phase array format for use in a skin assay is detailed in Section IVC.

IVA. Fluid-Phase Multiple Analyte Format

In this general approach, a sample containing one or more different-sequence heteroduplexes is first pretreated to remove interfering sample components, as described above with reference to FIG. 8A. It is also important, where heteroduplex discrimination is based on size, to treat the sample to remove ssRNA overhang, as discussed above.

The analytes shown in FIG. 8A include a plurality of different-sequence heteroduplexes, indicated H₁, H₂, and Hₙ, in the figure. Since the purpose of the initial solid-phase capture is to allow removal of unbound material, the binding agent used on the solid support must be specific for heteroduplex, but not for heteroduplex sequence. Preferably, the binding agent also shows little or no binding with free oligomer compound.

After washing the solid support to remove non-specifically bound material, the heteroduplexes are eluted, either as intact heterduplexes or as denatured single-strand oligomers, the later approach being accomplished by addition of denaturant or heat. The eluted heteroduplexes (or the corresponding oligomer compounds) are then collected, as in FIG. 8B and prepared for heteroduplex identification by separation of the eluted analytes.

In one method, illustrated in FIG. 8B, the eluted analytes are prepared for sequence analysis based on mass spectroscopy fragment analysis, according to methods and apparatus described, for example, in U.S. Pat. Nos. 5,770,859, 5,994,696, 5,770,858, and 5,827,659. FIG. 8C shows a hypothetical mass spectrum analysis of different-sequence oligomers, where the different peaks correspond to different sequence oligomers or oligomer fragments, and can be used to identify particular oligomer-compound sequences in sample.

In another general embodiment, different-sequence heterodimers or oligomer compounds are analyzed by gel electrophoresis, as illustrated in FIG. 8D, which shows a hypothetical electrophoretic pattern 100 obtained with a sample containing five different-sequence heterodimers, such as those indicated at 102, 104. The basis of the electrophoretic separation may be sequence-specific differences in size and/or charge. For example, oligomeric compounds with different numbers of bases, or different numbers of charged linkages, or different sizes of charged or uncharged polymer “tails”, or different numbers of charges in a polymer tail, each associated with a given oligomer base sequence, may be used in the composition administered to a subject.

Detection and/or identification of the separated bands may be made by one of a number of standard methods, including visualization with a colored or fluorescent nucleic-acid intercalating agents, elution and microsequencing, or elution and mass spec analysis.

IVB. Solid-Phase Multianalyte Detection

FIG. 9 shows a portion of an array device 110 used for detecting and identifying different sequence heteroduplexes, or oligomer compounds, in accordance with the invention. The device includes an array or assay regions, such as regions 112, 114, each having a sequence-specific binding agent (BA_{xy}) bound to the substrate surface in that region. For specific binding to sample heteroduplexes, the binding agents may be sequence-specific anti-heteroduplex antibodies, antigen-specific antibodies, or sequence-specific duplex binding agents, as described above with reference to FIGS. 6B, 6C, and 6E, respectively.

An advantage of the solid-phase array method is that sample clean-up and pretreatment may be avoided, since analyte binding to the regions of the array will be specific for both heteroduplexes and heteroduplex sequence. After exposing the array to the sample, under binding conditions, the array surface may be washed to remove non-specifically bound material, and then assayed for the presence of bound heteroduplex, e.g., by methods described with reference to FIGS. 7A-7C.

Thus, in one aspect, the invention includes an array device having a plurality of regions (or particles), each with a different binding agent capable of binding a different-sequence oligomer:RNA heteroduplex. Also included in the invention is a kit containing the array device and a detection agent for detecting the presence of heteroduplex bound to the device. The kit optionally contains an oligomer composition of the type described above, for administering to a subject.

FIG. 10 shows a hypothetical assay result on array device 110, employing the kit and method of the invention. The 8×8 array format assumes up to 64 different sequences, although some of the array regions will be devoted to controls and/or duplications. In the present format, the array results indicate detectable heteroduplex binding at four of the array regions, such as regions 112, 116. Using a key to the sequence carried at each array region, the user then knows that target RNA was present for four known sequences, which may be diagnostic of any of a variety of conditions discussed above.

IVC. Solid-Phase Skin Assay

In another embodiment, intended for either single- and multi-analyte testing, the oligomer or plurality of oligomers is administered transdermally. After a suitable period to allow for transdermal passage of the oligomer(s), entry of the oligomer(s) into cells below the skin surface, e.g., dendritic cells and subdermal skin cells, and formation and cellular expulsion of heteroduplex near the skin surface, the skin surface is then sample for the presence of heteroduplex. This is done by placing an adhesive tape over the skin region(s) to which the oligomer(s) were applied. The material collected in the adhesive is then released into a suitable aqueous medium for detection by any of the methods discussed above. The method relies on the ability of the administered oligomer(s) to be taken up by subdermal cells, and the localization of expelled heteroduplex in the region of skin administration.

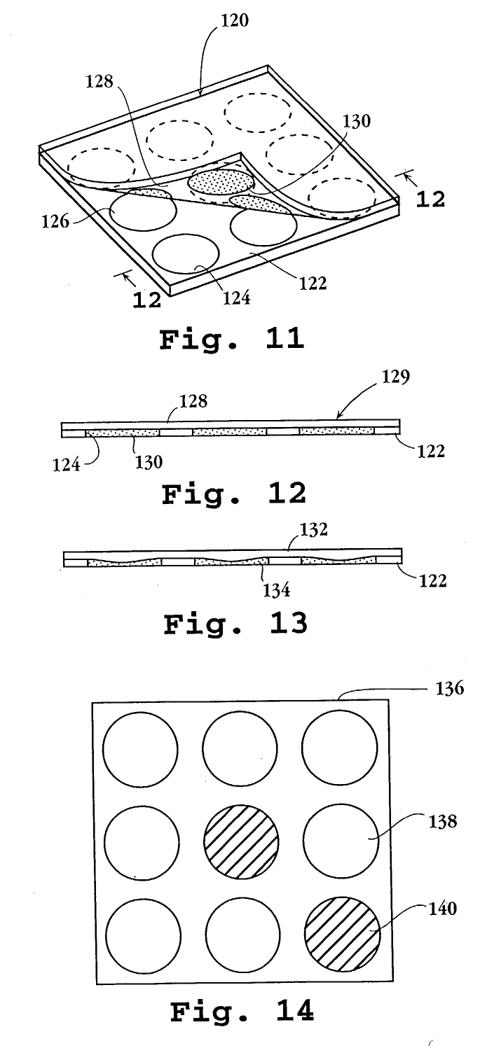

FIG. 11 shows an applicator 120 for use in administering a plurality of oligomers to a skin region of a patient. The applicator includes an adhesive patch 122 that is applied to the patient's skin area. The applicator patch has an array of openings, such as openings 124, 126 through which oligomer will be delivered to a selected skin region and through which heteroduplex will be collected. Carried over the applicator patch is an oligomer array layer 128 having an array of regions, such as region 130 in registry with corresponding openings in the applicator patch. Each region carries a selected oligomer in a suitable transdermal-delivery medium, such as a fluidic composition containing the oligomer, 50-90% propylene glycol, 5-10 percent linoleic acid or other long-chain fatty acid, and remainder water. To keep the regions in a moist condition prior to skin application, the lower surface of the patch is covered with a film that is removed shortly before applicator use.

When the applicator is placed on a patient skin surface, oligomers from each of the array regions of the applicator are brought into contact with the skin surface, as seen FIG. 12, allowing oligomers in the array regions to be administered transdermally to the patient. The period of administration, i.e., the period during which layer 128 is held in contact with the skin, is typically 1-4 hours, after which the layer is removed from the patch, which is retained on the patient skin surface.

To collect sample, a collector layer 132 having an adhesive backing 134 is placed over patch 122, adhesive side down, bringing the adhesive into contact with the skin in the areas of patch openings, as seen in FIG. 13. The adhesive, which is typically a tacky polymer type adhesive is effective to bond to the upper surface layer of the skin. Removal of the collector layer from the patch is thus effective to collect cells, dermal debris, and any heteroduplex contained in the upper dermal layer. The collector layer now forms an array of adhesive regions, each having dermal material collected through one of the patch openings.

Because heteroduplex that is formed in the method will remain relatively localized at the site of administration, heteroduplex contained on the collector layer will correspond to the particular oligomer administered at the same skin region.

To detect heteroduplex collected on the collector layer, the layer is placed over a multi-well plate, such as plate 136 seen in FIG. 14, having wells, such as wells 138, 140 disposed in registry with the collection regions on the collector layer. When the layer is placed on the top surface of plate 136 it forms an adhesive seal between the plate and layer, with the collection regions exposed to the open wells in the plate. These wells are filled with a suitable extraction medium, e.g., an aqueous surfactant medium designed to dissolve or partially dissolve the adhesive, with release of material trapped in the adhesive into the medium. The transfer may be accomplished by pressing the adhesive regions down into contact with the extraction medium contained the corresponding wells, or by agitating plate 136, or by turning the plate over, with the collector plate down. After a suitable extraction period, e.g., 30-60 minutes at room temperature, the collector layer is removed from the plate to expose the wells and solutions therein.

Heteroduplex in any of the arrays is detected by any of the methods detailed above, such as capture of heteroduplex on the surface of the wells through a heteroduplex binding agent, and subsequent detection of bound heteroduplex using a reporter-labeled heteroduplex binding agent.

The following examples illustrate but are not intended in any way to limit the invention.

### EXAMPLE 1

Formation of Nuclease-Resistant Antisense Oligo:RNA Heteroduplexes in vitro and in vivo

In Vitro Studies

Duplex formation was evaluated by mixing various mRNAs with antisense oligomers, allowing them to hybridize followed by visualization of duplex formation on 12% non-denaturing acrylamide gels run at 36 V for 4.75 hours and stained with ethydium bromide to detect duplex formation and RNAse resistance. The migration of the oligonucleotides in the gel is based on charge to mass and in the case of duplexes, the mass is nearly double that of the RNA alone but no charge is added as the PMO is neutral. The migration of the duplex varies with the acrylamide gel concentration.

An alpha globin synthetic mRNA 25-mer (SEQ ID NO:1) and a non-complementary PMO oligomer antisense to c-myc (SEQ ID NO:2), or a complementary, alpha globin antisense PMO 25-mer (SEQ ID NO:3) were mixed in the presence or absence of RNAse.

When the alpha globin synthetic 25-mer was mixed with a non-complementary PMO 25-mer having a sequence antisense to c-myc (PMO 122-126, SEQ ID NO:2), only a single band was observed following gel electrophoresis and the molecular weight of the band was consistent with that of the synthetic mRNA 25-mer. However, when the alpha globin synthetic 25-mer was mixed with a complementary, alpha globin antisense PMO 25-mer (SEQ ID NO:3), two bands were observed following gel electrophoresis, a lower band migrating at the predicted rate for the mRNA 25-mer plus a second band migrating at rate predicted for an oligomer of about 200-base pairs. The upper band, but not the lower band, was resistant to treatment with RNAseBM or RNAseT1 prior to loading.

The results indicated that an RNAse resistant duplex was formed between an alpha globin synthetic mRNA 25-mer (SEQ ID NO:1) and a complementary antisense PMO (SEQ ID NO:3) in the presence of RNAseBM, as indicated by a faint band at the expected gel migration point for a PMO:RNA duplex and no band for the RNA alone.

The results further indicated that an RNAse resistant duplex was formed between an alpha globin synthetic mRNA 25-mer (SEQ ID NO:1) and a complementary antisense PMO (SEQ ID NO:3) in the presence of RNAseT1, as indicated by a band at the expected gel migration point for a PMO:RNA duplex and no band for the RNA alone, indicating the RNA can be degraded when not part of the duplex.

A comparison of the results of electrophoresis with mixtures of complementary versus non-complementary mRNA:antisense oligomer pairs confirmed that a duplex forms between mRNA and its complementary antisense PMO oligomer, that the duplex is resistant to degradation by RNAse. The relative gel electrophoresis migration rate of mixtures of complementary mRNA:antisense oligomer pairs in the presence and absence of RNAse, show that a duplex forms between an alpha globin synthetic mRNA 25-mer (SEQ ID NO:1) and a complementary antisense PMO (SEQ ID NO:3) and that excess alpha globin synthetic mRNA is present in the absence of RNAse.

In Vivo Studies

Antisense oligomers were injected intraperitoneally into rats followed by formation of stable oligomer:RNA heteroduplexes in vivo which were subsequently detectable in rat urine.

For each test animal, one ml of urine collected 24 hours following administration, was dialyzed against a standard assay buffer in 6000 to 8000 mw cutoff dialysis tubing (Spectra/Por) to remove salts. The dialyzed samples were incubated with DNAse and RNAses for 10 minutes and dried in a Savant Speed-Vac. Dried samples were dissolved in 50 μl water and 25 μl was loaded per lane onto a 12% non-denaturing acrylamide gel.

Rats were administered saline, or 3 nmoles, 75 nmoles or 375 nmoles of the PMO 122-126 25-mer antisense to c-myc (SEQ ID NO:2) at the time of partial hepatectomy. The results of gel electrophoresis show the presence of a DNAse and RNAse-resistant band which migrates near the 200 bp DNA ladder band, consistent with that of a PMO:RNA heteroduplex. Appearance of this band is dependent on the amount of PMO administered, and is absent when rats are injected with saline. In rats given 375 nmoles of the PMO 122-126 25-mer antisense to c-myc (SEQ ID NO:2) at the time of partial hepatectomy a band is observed which is consistent with the migration pattern of a PMO:RNA duplex, which supports the detection of a PMO:RNA duplex following in vivo exposure to the PMO.

These observations support the formation in vivo of a specific, detectable antisense oligomer:RNA heteroduplex upon administration of a PMO to an animal. This heteroduplex forms intracellularly and remains resistant to nucleases and stable to changes in osmolality throughout its transit through the cell membrane into the renal blood supply, its clearance through the kidneys into the urine.

### EXAMPLE 2

In Vivo Studies with Antisense Oligomer:RNA Heteroduplexes

Calibration studies performed using an instrument capable of detecting fluorescein conjugated oligomers (Applied Biosystems Model 672 GeneScanner) were used to determine the migration rates of fluorescein-conjugated oligomers of various lengths; a 15-mer, a 20-mer, a 24-mer and a 38-mer ribozyme. Migration rates were evaluated on a GeneScanner gel and calibration studies confirmed the validity of the GeneScanner approach to detection of PMO:RNA duplexes. Calibration studies show that the Applied Biosystems Model 672 GeneScanner can distinguish fluorescein conjugated oligomers on the basis of both length and concentration.

In Vivo Studies

Rats were injected with a carboxyfluorescein-conjugated PMO (SEQ ID NO:5), which is antisense to rat cytochrome P-4503A2 (SEQ ID NO:6).

GeneScanner chromatograms of plasma samples prepared from blood withdrawn from rats one hour post-injection contained fluorescent components which migrated at 270 and 340 minutes (two peaks due to the two possible carboxyfluorescein linkages which migrate differently). Plasma samples prepared from rats 24 hours post-injection contained fluorescent components which migrated at approximately 75 and 80 minutes. Mass spectral data (not shown) confirms that the shorter migration time is not due to degradation of the PMO and indicates that a PMO:RNA heteroduplex has been formed over that time.

FIG. 2 represents the results of an analysis of samples taken at various times post administration of the P450 antisense PMO, and indicates the disappearance of the PMO monomer and the corresponding appearance of PMO:RNA heterodimer in the plasma of rats following such administration. Appearance of significant quantities of the duplex in plasma does not occur until the majority of the unduplexed PMO leaves the plasma in what is generally referred to as the “distribution phase”. The PMO heteroduplex does not accumulate in plasma until after PMO monomer has distributed into the tissues of the subject where the complementary mRNA transcripts are localized. The charged PMO:RNA heteroduplex presumably forms in these tissues and effluxes out of cells and back into plasma. This overall process requires several hours.

After administration of the p450 antisense PMO (SEQ ID NO:5), fluorescein was detected in both the kidney and liver.

Chromatograms of kidney tissue samples showed a band at 350 minutes consistent with unduplexed PMO and an additional band at 80 minutes consistent with the PMO:RNA heteroduplex, indicating both duplex and parent PMO which may reside in interstitial spaces or within the cells of the kidney. The liver tissue sample showed essentially no unduplexed PMO and significantly more PMO:RNA heteroduplex. These results are consistent with the observation that levels of P450 mRNA transcript are much lower in kidney than in liver.

Studies reflecting the time course of urinary clearance of unduplexed antisense PMO oligomer and antisense PMO oligomer:RNA heteroduplexes indicate that several hours are required for formation and efflux of PMO:RNA heteroduplex from tissues into plasma, followed by their ultimate appearance in urine.

### EXAMPLE 3

### Development of Multiple Monoclonal Antibodies that Recognize the Phosphorodiamidate Morpholino Oligomers (PMO).

The following details the preparation of multiple monoclonal antibodies that recognize the phosphorodiamidate morpholino oligomers of the present invention. To form the immunogen, the 5′-end of a PMO was linked to keyhole limpet hemocyanin (KLH) by standard linker chemistry. The PMO-KLH conjugate was hybridized to complementary RNA, then injected into mice, followed by boosting and bleeding the mice to determine whether strong antibody titer to PMO existed.

In mice in which a strong antibody response was observed, spleens were removed and isolated spleen cells were fused with an immortalizing cell line to prepare hybridomas, according to well known methods. Among the cell lines screened, ten were observed that that secrete antibodies that recognize the PMO. From these ten, three general monoclonal antibody (MAb) recognition types were isolated; (a) three of the ten clones secreted Mab's which recognize the triethyleneglycol moiety conjugated to the 5′-end of PMO, and (b) seven of the ten clones had Mab's which recognize the PMO heteroduplex structure. Of these seven, six lines produced Mab's that do not appear to recognize a unique sequence of the PMO, but do not recognize RNA or DNA. One of the seven Mab's which recognized the PMO is also substantially more sensitive to the sequence of the particular PMO used to immunize than other PMO sequences.

Polyclonal serum was also evaluated for detecting the PMO in the vascular wall from pigs injected with a PMO of the present invention by an infiltrating catheter adapted for vascular delivery. Tissue lysate of coronary vessels were loaded into an acrylamide gel, an electric field was applied, then the gel contents were transferred to a Nytran membrane following the method of a western blot. The membrane was probed with PMO anti-sera and bands were visible for free PMO which did not migrate in the gel and another band visible which is the RNA:PMO duplex which moves in the gel due to the negative charge associated with the RNA.

Although the invention has been described with reference to specific methods and embodiments, it will be appreciated that various modifications and changes may be made without departing from the invention.

SEQUENCE LISTING TABLE

| Description | SEQ ID NO |
|---|---|
| synthetic 25-mer corresponding to alpha | 1 |
| globin mRNA | |
| (5′-CGA GUC CGU CUG AGA AGG AAG GAG G-3′) | |
| PMO 25-mer antisense to c-myc (nt | 2 |
| 1-22-126; | |
| 5′-ACGTIGAGGGGCATCGTCGC-3′) | |
| PMO 25-mer antisense to alpha globin mRNA | 3 |
| PMO antisense to rat cytochrome P-4503A2 | 4 |
| (1-0-256; | |
| 5′-UGA GAG CUG AAA GCA GGU CCA U-3′) | |
| Carboxyfluorescein conjugated PMO | 5 |
| complementary (antisense) to rat | |
| cytochrome P-4503A2 (1-0-256) | |
| rat cytochrome P-4503A2 | 6 |

It is claimed:

1. A method of detecting in a subject, the occurrence of a base-specific intracellular binding event involving a single-stranded target RNA, comprising
(a) administering to the subject an oligomeric antisense compound having (i) from 8 to 40 bases, including a targeting base sequence that is complementary to a portion of the target RNA, (ii) a Tm, with respect to binding to a complementary RNA sequence, of greater than about 50° C., and (iii) an ability to be actively taken up by mammalian cells, and (iv) conferring resistance of complementary RNA hybridized with the agent to RNaseH,
(b) at a selected time after said administering, obtaining a sample of a body fluid from the subject, and
(c) detecting in the sample the presence of a nuclease-resistant heteroduplex composed of the antisense oligomer and the complementary portion of the target RNA.

2. The method of claim 1, wherein the antisense compound has a substantially uncharged backbone.

3. The method of claim 2, wherein the antisense compound is a morpholino antisense compound having uncharged, phosphorous-containing intersubunit linkages.

4. The method of claim 1, wherein said detecting includes capturing the heteroduplex on a solid support, by binding to a support-bound capture agent capable of binding heteroduplex but not free antisense agent, and detecting heteroduplex so captured.

5. The method of claim 4, where the capture agent is selected from the group consisting of (a) an antibody capable of binding in a sequence-independent manner to the heteroduplex, (b) an antibody capable of binding in a sequence-dependent manner to a heteroduplex in a sequence-dependent manner), (c) an antibody capable of binding to an antigen attached to the antisense compound, (d) a non-antibody antiligand molecule capable to binding to a ligand moiety attached to the antisense compound, and (e) a base-specific duplex-binding oligomer.

6. The method of claim 4, wherein said detecting includes contacting the solid support and bound heteroduplex with a detection reagent selected from the group consisting of of (a) a labeled antibody capable of binding to the heteroduplex, (b) a labeled antibody capable of binding to an antigen attached to the antisense compound, (c) a labeled non-antibody antiligand molecule capable to binding to a ligand moiety attached to the antisense compound, (d) a labeled duplex-binding oligomer, and (e) a labeled cationic polymer.

7. The method of claim of claim 4, wherein said detecting includes eluting heteroduplex bound to the support, and detecting eluted heteroduplex.

8. The method of claim 1, for use in detecting changes in expression of a target gene in response to a therapeutic agent administered to the subject, wherein the target RNA is mRNA produced by expression of the target gene, steps (a)-(c) are performed at selected times before and administration of the therapeutic agent, and said detecting includes comparing the levels of heteroduplex detected before and after such administration.

9. The method of claim 1, for use in detecting the presence or levels of an mRNA which is diagnostic of a given biochemical or pathological state or a predisposition to such state selected from the group consisting or (i) pregnancy, (ii) heart disease, (iii) alcoholism, and (iv) cancer, wherein the target RNA is an mRNA encoding a protein selected from the group consisting of (i) hCG, (ii) [heart-disease markers], (iii) [alcoholism markers], and (iv) [cancer markers].

10. The method of claim 1, for use in detecting the presence of a mutated gene which is diagnostic of a given genetic disease, wherein the target RNA is an mRNA transcribed by the gene and encodes a mutated protein selected from the group consisting of [selected from the group consisting of [known mutated proteins for various genetic diseases], and the antisense compound target.

11. The method of claim 10, wherein the antisense compound is designed to form a stable heteroduplex above 50° C. only with the mutated form of the mRNA, and said detecting may optionally include heating heteroduplex in the sample above 50° C. to denature heteroplexes with one or more internal-base mismatches.

12. The method of claim 1, for use in detecting the presence of an infective viral or bacterial agent in the subject, wherein the target RNA is a single-stranded RNA or DNA having a virus-specific or bacteria-specific sequence, respectively.

13. The method of claim 1, wherein said administering includes applying the antisense agent to a region of the subject's skin, said obtaining includes applying an adhesive tape to said skin region, and said detecting includes detecting the presence of heteroduplex on the adhesive tape.

14. A method of detecting in a subject, the occurrence of base-specific intracellular binding events involving a plurality of target RNAs, comprising
(a) administering to the subject a plurality of different-sequence oligomeric antisense compounds, each having (i) from 8 to 40 bases, including a targeting base sequence that is complementary to a portion of an RNA transcript produced by a selected one of a plurality of target genes, (ii) a Tm, with respect to binding to a complementary RNA sequence, of greater than about 50° C., and (iii) an ability to be actively taken up by mammalian cells, and (iv) conferring resistance of complementary RNA hybridized with the agent to RNaseH,
(b) at a selected time after said administering, taking a sample of a body fluid from the subject, and
(c) detecting in the sample the presence of a nuclease-resistant heteroduplexes, each composed of the antisense oligomer and the complementary portion of the corresponding RNA transcript.

15. The method of claim 14, wherein the antisense compound has a substantially uncharged backbone.

16. The method of claim 15, wherein the antisense compound is a morpholino antisense compound having uncharged, phosphorous-containing intersubunit linkages.

17. The method of claim 14, wherein said detecting includes capturing the heteroduplex species on a solid support having an array of regions, where each region contains a sequence-specific support-bound capture agent capable of specifically binding to a heteroduplex species of a selected sequence, and identifying array regions having bound heteroduplex species.

18. The method of claim 17, wherein said capture agents are selected from the group consisting of (a) an antibody capable of binding in a sequence-dependent manner to a heteroduplex in a sequence-dependent manner), (b) an antibody capable of binding to an antigen attached to an associated antisense compound, where the antisense agent in each different-sequence heteroduplex species has a unique antigen, and (c) a base-specific duplex-binding oligomer of effective to capture a specific-sequence heteropuplex.

19. The method of claim 14, wherein said sample contains a plurality of different-sequence heteroduplexes, each having a different molecular weight and/or charge, and said detecting includes identifying the different the heteroduplexes by mass spectroscopy or electrophoresis.

20. The method of claim 19, wherein said detecting includes partially purifying heteroduplexes from said sample by affinity binding of different-sequence heteroduplexes to a solid support having a support-bound binding agent effective to bind heteroduplexes, but not the antisense agent alone, and eluting the bound heteroduplexes from the solid support.

21. The method of claim 14, for use in detecting one of a plurality of different known-mutation gene sequences associated with one or more known disease states, wherein the target RNAs are mRNA's transcribed by the gene sequences and encodes a mutated proteins selected from the group consisting of [selected from the group consisting of [known mutated proteins for various genetic diseases], and the antisense compound target.

22. The method of claim 14, for use in detecting the presence of one or more of a plurality of different viruses or bacteria, where steps (a)-(c) are carried out successively with (i) first and second sets of antisense agents effective to bind to viral or bacterial sequences representing relatively broad and relatively narrow classes of viruses or bacteria, and the second set of antisense agents is selected on the basis of the heteroduplex(es) formed and detected using the first set of agents.

23. The method of claim 14, wherein said administering includes applying to a the subject's skin, an adhesive pad containing a lower adhesive layer adapted to be attached adhesively to the subsjects skin, and defining an array of holes adapted to expose an array of skin regions, and a removable antisense delivery layer containing an array of different-sequence antisense agents at positions corresponding to said lower-layer holes, for administering the antisense agents transdermally to the subject when the adhesive pad is applied to the subject's skin, and said detecting includes removing said delivery layer, replacing it with an adhesive sample-collection layer, thereby to collect sample on the adhesive layer at array regions corresponding to said holes, and detecting the presence of heteroduplex at such array regions on the sample-collection layer.

24. An diagnostic array device for use in a subject, the occurrence of base-specific intracellular binding events involving a plurality of target RNAs, comprising
a substrate divided into a plurality of regions, and
(d) carried on each array region, a sequence-specific binding agent capable of binding to a specific-sequence heteroduplex composed of an RNA oligomer of a specific sequence and a complementary-sequence antisense oligomer characterized by (i) a Tm, with respect to binding to the complementary RNA oligomer, of greater than about 50° C., and (iii) an ability to be actively taken up by mammalian cells, and (iv) conferring resistance of complementary RNA hybridized with the agent to RNaseH,
where each of said binding agents is selected from the group consisting of (a) an antibody capable of binding in a sequence-dependent manner to a heteroduplex in a sequence-dependent manner, (b) an antibody capable of binding to a sequence-specific antigen attached to the antisense compound, and (c) a sequence-specific duplex-binding oligomer.

25. The array of claim 24, wherein the sequence-specific binding agent is capable of sequence-specific binding to such a heteroduplex in which the antisense agent has a substantially uncharged backbone.

26. A kit for use in detecting in a subject, the occurrence of base-specific intracellular binding events involving a plurality of target RNAs, comprising
the array device of claim 24, and
a detection reagent capable of binding to such heteroduplex species bound to one or more regions of the array.

27. The kit of claim 26, wherein the detection reagent is selected from the group consisting of (a) a labeled antibody capable of binding in a sequence-independent or sequence-dependent manner to the heteroduplex, (b) a labeled antibody capable of binding to an antigen attached to the antisense compound, (c) a labeled non-antibody antiligand molecule capable to binding to a ligand moiety attached to the antisense compound, (d) a labeled duplex-binding oligomer, and (e) a labeled cationic polymer.

28. The kit of claim 26, wherein the sequence-specific binding agent in the array device is capable of sequence-specific binding to such a heteroduplex in which the antisense agent has a substantially uncharged backbone.

29. A monoclonal antibody having specific binding affinity for a heteroduplex composed of an RNA oligomer and a complementary-sequence antisense oligomer characterized by (i) a substantially uncharged backbone, (ii) a Tm, with respect to binding to the complementary RNA oligomer, of greater than about 50° C., and (iii) an ability to be actively taken up by mammalian cells, and (iv) conferring resistance of complementary RNA hybridized with the agent to RnaseH.

30. The antibody of claim 29, whose binding affinity for the heteroduplex is substantially independent of heteroduplex sequence.

31. The antibody of claim 29, whose binding affinity for the heteroduplex is substantially dependent on heteroduplex sequence.
